(19) United States
(12) Patent Application Publication
(10) Pub. No.: US 2025/0228834 A1
(43) Pub. Date: Jul. 17, 2025

(54) PHARMACEUTICAL COMPOSITION FOR THE TREATMENT OF COLON AND LUNG CANCER

(71) Applicant: HEPAREGENIX GMBH, Tübingen (DE)

(72) Inventor: Rene BERNARDS, Tübingen (DE)

(21) Appl. No.: 18/854,384

(22) PCT Filed: Apr. 5, 2023
(86) PCT No.: PCT/EP2023/058953

§ 371 Date: Oct. 4, 2024

(30) Foreign Application Priority Data
Apr. 6, 2022 (EP) 22166913.8

(51) Int. Cl. A61K 31/444; A61K 31/496; A61K 31/506; A61K 31/519; A61K 45/06

(52) U.S. Cl. CPC A61K 31/444; A61K 31/496; A61K 31/506; A61K 31/519; A61K 45/06

(57) ABSTRACT

The present invention relates to a pharmaceutical composition for the treatment of colon and lung cancer comprising a MKK4 inhibitor and a MEK, ERK, KRAS or SHP2 inhibitor. The composition provides a strong synergistic effect in the treatment of colon and lung cancer and in particular in the treatment of KRAS mutant colon and lung cancer.

The present invention relates to a pharmaceutical composition for the treatment of colon and lung cancer comprising a MKK4 inhibitor and a MEK, ERK, KRAS or SHP2 inhibitor.

### BACKGROUND OF THE INVENTION

The treatment of cancer is gradually changing from an organ-centered to a pathway-centered approach. In recent years, significant progress has been made in the treatment of both common and rare cancers. This progress is leading to longer patient survival and improved quality of life. Advances range from targeted therapies for disease settings where previously no effective treatments existed to exciting progress in immunotherapy. The focus of cancer treatment is shifting toward targeted therapies aimed at genes and pathways involved in human cancer. Targeted therapy is a treatment that targets specific molecules in or on cancer cells, or in the tumor's immediate surroundings. This type of approach is aimed at blocking the growth and spread of cancer cells while limiting damage to healthy cells. Important molecular targets in targeted therapy include components of signaling pathways. Signaling pathways normally connect extracellular signals to the nucleus leading to expression of genes that directly or indirectly control cell growth, differentiation, survival, and death. For many cancers it has been established that signaling pathways are dysregulated. The dysregulated signaling pathways may be linked to tumor initiation and/or progression. Targeting such dysregulated pathway may thus provide a beneficial treatment option. Despite recent advances in understanding mechanisms involved in cancer, targeted therapy is not always successful. For example, one signaling pathway implicated in human oncogenesis is the RAS-RAF-MEK-ERK or MAPK pathway. BRAF (proto-oncogene B-Raf or v-Raf murine sarcoma viral oncogene homolog 1) and KRAS (Kirsten rat sarcoma viral oncogene homologue) are two key oncogenes in the RAS/RAF/MEK/MAPK signaling pathway. Numerous efforts to develop therapeutic agents that specifically target the mutated BRAF kinase are underway for melanoma treatment. However, the development of resistance to the BRAF inhibitors has proven to be a major challenge.

Another example of the challenges faced in the field relate to MEK inhibition in KRAS mutant lung and colon cancer. Several compounds with potent inhibitory activity specific for MEK1/2 have been investigated. The first MEK inhibitor entered clinical trials in 2000, but until 2014 no MEK inhibitor had been approved for clinical use. This is because, for the most part, the agents investigated have not demonstrated robust clinical activity in most tumor types. One of the possible mechanism by which a cancer becomes or is unresponsive to a MEK inhibitor is based on acquiring or inherently possessing resistance to the MEK inhibitor.

Activating mutations in KRAS is present in over 40% of non-small-cell lung cancer (NSCLC) and 50% of colorectal cancer (CRC) and is one of the most common oncogenic drivers in human cancers, see Mutations in Human Cancers Related to KRAS Update. (2021, January 30), National Cancer Institute; https://www.cancer.gov/research/key-initiatives/ras/ras-central/blog/2021/update-kras-cancer-comutations. Activating KRAS mutations promote and maintain tumorigenesis and correlates with a very poor prognosis, ultimately contributing to over one million deaths per year worldwide, Wang et al., Biochim Biophys Acta. 2007 August; 1773(8):1248-55. Despite drug-targeting efforts for the past four decades, with the exception of the mutant specific KRASG12C inhibitor, no selective and targeted drugs have been developed that effectively target KRAS mutants. Due to the “undruggable” nature of KRAS, many efforts have been focused on inhibition of the RAF-MEK-ERK (MAPK) pathway downstream. Unfortunately, MEK inhibitors showed poor clinical responses, Wang et al., Biochim Biophys Acta. 2007 August; 1773(8):1248-55 and Janne, Pasi A et al., JAMA vol. 317, 18 (2017): 1844-1853.

The reactivation of KRAS and its downstream effectors during drug treatment has shown to be one of the mechanisms of drug resistance, Xue et al., Cell Res. 2018 July; 28(7):719-729 and Sun et al., Cell Rep. 2014 Apr. 10; 7(1):86-93. MEK and ERK inhibitors activate the MAP2K4-JNK-JUN pathway, leading to activation of HER Receptor Tyrosine Kinases (RTKs) and thereby reactivating the MAPK pathway upon its initial inhibition. Despite recent advances in understanding mechanisms involved in cancer and in diagnosis and treatment, drug therapies seldom offer a long-term cure.

SHP2 (Src homology-2 domain-containing protein tyrosine phosphatase-2), an oncogenic tyrosine phosphatase involved in signal transduction downstream of several RTKs, has been associated with several types of cancer. These include leukemia and breast, gastric, laryngeal, liver, lung, and oral cancers, as well as other diseases. Acting upstream of RAS, SHP2 is necessary for full activation of the MAPK pathway. SHP2 is a well-validated PTP oncoprotein in humans and is emerging as an important target for cancer treatment. Currently, two types of small molecules inhibiting SHP2 activity have been reported. Type I SHP2 inhibitors interact specifically with the PTP catalytic pocket; type II SHP2 inhibitors (or allosteric inhibitors) bind to a region outside of the PTP catalytic pocket and exhibit higher selectivity against other members in the phosphatase family.

Accordingly, the technical problem underlying the present invention can been seen in the provision of products, compositions, methods and uses for achieving a therapeutic benefit for cancer patients. The problem underlying the invention is in particular to enable effective treatment of colon and lung cancer, in particular KRAS mutant colon and lung cancer.

### SUMMARY OF THE INVENTION

The problem underlying the invention is solved by providing a pharmaceutical composition comprising a) a MKK4 inhibitor which is selective over MKK7, JNK1 and BRAF and b) a MEK, ERK, KRAS or SHP2 inhibitor.

### BRIEF DESCRIPTION OF FIGURES

FIG. 1 presents the colony formation matrices, which illustrates the effect of compound (A) and the MEK-inhibitor trametinib alone on the growth of cancer cell lines with different KRas^{G12C} and KRas^{G13D}-mutations and the synergistic action of the combination of both drug substances.

FIG. 2 presents the colony formation matrices, which illustrates the effect of compound (A) and the ERK-inhibitor SCH772984 alone on the growth of cancer cell lines with different KRas^{G12C} and KRas^{G13D}-mutations and the synergistic action of the combination of both drug substances.

FIG. 3 presents the colony formation matrices, which illustrates the effect of compound (A) and the KRas^{G12C}-inhibitor AMG510 (sotorasib) alone on the growth of cancer cell lines with different KRas^{G12C} and KRas^{G13D}-mutations and the synergistic action of the combination of both drug substances.

FIG. 4 presents the colony formation matrix, which illustrates the effect of compound (A) and the SHP2-inhibitor RMC4550 alone on the growth of a cancer cell line H358 bearing a KRas^{G12C}-mutation and the synergistic action of the combination of both drug substances.

FIG. 5 illustrates the synergistic action of compound (A) in combination with MEK, SHP2 and KRASG12C inhibitors. (A) Confluence and (B) Caspase 3/7 activity

### DETAILED DESCRIPTION OF THE INVENTION

The invention relates to the following embodiments:
1. A pharmaceutical composition comprising a component a) and a component b), wherein
component a) comprises at least one compound having formula (I)

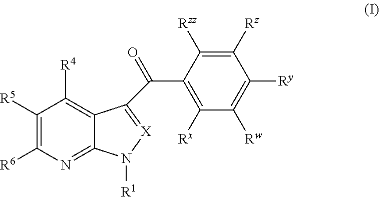

wherein
X is —CR² or N;
R¹ is H or alkyl;
R² is H or alkyl;
R⁴ is H, or alkyl;
R⁶ is H, or alkyl;
R^{w} is —NR¹⁰SO₂R¹²;
R¹⁰ is H, alkyl, or phenylalkyl;
R¹² is alkyl or haloalkyl;
R^{x}, R^{y}, R^{z} and R^{zz} are selected from:
a) R^{x} and R^{y} are halogen and R^{z} and R^{zz} are H;
b) R^{x}, R^{y} and R^{zz} are independently halogen and R^{z} is H;
c) R^{x}, R^{y} and R^{z} are independently halogen and R^{zz} is H;
R⁵ is selected from
   (a) phenyl which is substituted with 1, 2 or 3 groups independently selected from
      halogen,
      alkyl,
      alkoxy,
      alkoxy wherein the alkyl group is substituted with 1, 2 or 3 hydroxy groups,
      hydroxy,
      —SO₂NR¹⁰R¹⁰,
      —CO₂R¹⁰,
      —CN,
      —SF₅,
      —(NR¹⁰═)S(═O)-alkyl (S-alkylsulfonimidoyl),
      1H- or 2H-tetrazolyl,
      —POdi(alkyl),
      R¹⁰R¹⁰N—CO—,
      hydroxyalkyl-ONH—CO—,
      —COO—CH₂CH₂—CH(NH₂)—COOR¹⁰,
      —OCH₂O— (methylenedioxy attached in neighboring positions to the phenyl ring),
      —OCH₂CH₂O— (ethylenedioxy attached in neighboring positions to the phenyl ring),
      a non-aromatic heterocyclic 5- or 6-membered monocyclic group having 1, 2 or 3 heteroatoms independently selected from 0 and N;
   (b) naphthyl;
   (c) a heteroaromatic 5- or 6-membered monocyclic group having 1, or 2 heteroatoms independently selected from O, N and S, wherein the heteroaromatic group is optionally substituted with 1, 2 or 3 groups independently selected from
      alkyl,
      haloalkyl,
      cycloalkyl,
      —NR¹⁰R¹⁰,
      halogen,
      alkoxy, which is optionally substituted with —NR¹⁰R¹⁰
      —CN,
      alkenyl,
      alkinyl,
      R¹⁰R¹⁰N—CO—,
      —(NR¹⁰═)S(═O)-alkyl,
      cycloalkyl-NR¹⁰—,
      alkyl-NR¹⁰—, wherein the alkyl group is substituted with hydroxy or alkoxy,
      alkylsulfanyl,
      —COOR¹⁰,
      1H- or 1H tetrazolyl, and
      a non-aromatic heterocyclic 4-, 5- or 6-membered monocyclic group having 1 or 2 heteroatoms independently selected from 0 and N, which heterocyclic group is optionally substituted with alkyl, hydroxyalkyl or hydroxy,
   (d) phenyl which is fused with a heteroaromatic 5- or 6-membered monocyclic group having 1, 2 or 3 heteroatoms independently selected from O, N and S;
or a pharmaceutically acceptable salt, solvate or optical isomer thereof; and
component b) comprises at least one inhibitor selected independently from
   b1) a MEK inhibitor,
   b2) an ERK inhibitor,
   b3) a KRAS, in particular a KRAS^{G12C} inhibitor, and
   b4) a SHP2 inhibitor;
or a pharmaceutically acceptable salt, solvate or optical isomer thereof.
2. A composition of embodiment 1, wherein component a) comprises a compound of formula (I), wherein R¹, R², R⁴ and R⁶ are H; or a pharmaceutically acceptable salt, solvate or optical isomer thereof.
3. A composition of embodiment 1 or 2, wherein component a) comprises a compound of formula (I), wherein R^{w} is —NHSO₂R¹²; or a pharmaceutically acceptable salt, solvate or optical isomer thereof.
4. A composition of embodiment 3, wherein R¹² is alkyl, in particular C₁-C₄ alkyl, such as methyl, ethyl, n-propyl, isopropyl or n-butyl.
5. A composition of any one of the preceding embodiments, wherein component a) comprises a compound of formula (I), wherein R^{x}, R^{y}, R^{z} and R^{zz} are selected from
a) R^{x} and R^{y} are halogen and R^{z} and R^{zz} are H; and
b) R^{x}, R^{y} and R^{zz} are independently halogen and R^{z} is H;
or a pharmaceutically acceptable salt, solvate or optical isomer thereof.
6. A composition of any one of the preceding embodiments, wherein R^{x} and R^{y} are F and R^{z} and R^{zz} are H.
7. A composition of any one of embodiments 1 to 4, wherein R^{x}, R^{y} and R^{zz} are F and R^{z} is H or R^{x}, R^{y} and R^{z} are F and R^{zz} is H.
8. A composition of any one of the preceding embodiments, wherein component a) comprises a compound of formula (I), wherein X is —CR²; or a pharmaceutically acceptable salt, solvate or optical isomer thereof.
9. A composition of embodiment 8, wherein R⁵ is selected from
   (a) phenyl which is substituted with 1, 2 or 3 groups independently selected from
      halogen,
      alkyl,
      alkoxy,
      alkoxy wherein the alkyl group is substituted with 1, 2 or 3 hydroxy groups,
      hydroxy,
      —SO₂NR¹⁰R¹⁰,
      —CO₂R¹⁰,
      —CN,
      —SF₅,
      —(NR¹⁰═)S(═O)-alkyl (S-alkylsulfonimidoyl),
      1H- or 2H-tetrazolyl, and
      —PO(dialkyl), and
   (b) naphthyl;
   (c) a heteroaromatic 5- or 6-membered monocyclic group having 1, or 2 heteroatoms independently selected from O, N and S, wherein the heteroaromatic group is optionally substituted with 1, 2 or 3 groups independently selected from
      alkyl,
      cycloalkyl,
      —NR¹⁰R¹⁰
      halogen,
      alkoxy, which is optionally substituted with —NR¹⁰R¹⁰
      —CN,
      alkenyl,
      R¹⁰R¹⁰N—CO—,
      —(NR¹⁰═)S(═O)-alkyl,
      cycloalkyl-NR¹⁰—,
      alkyl-NR¹⁰—, wherein the alkyl group is substituted with hydroxy or alkoxy, alkylsulfanyl, and
   (d) phenyl which is fused with a heteroaromatic 5- or 6-membered monocyclic group having 1, 2 or 3 heteroatoms independently selected from O, N and S.
10. A composition of embodiment 9, wherein R⁵ is selected from
   (a) phenyl which is substituted with 1, 2 or 3 groups independently selected from
      halogen,
      alkyl,
      alkoxy,
      alkoxy wherein the alkyl group is substituted with 1, 2 or 3 hydroxy groups,
      hydroxy,
      —SO₂NR¹⁰R¹⁰,
      —CO₂R¹⁰,
      —CN,
      —SF₅,
      —(NR¹⁰═)S(═O)-alkyl (S-alkylsulfonimidoyl),
      1H- or 2H-tetrazolyl, and
      —PO(dialkyl) and
   (b) a heteroaromatic 5- or 6-membered monocyclic group having 1, or 2 heteroatoms independently selected from O, N and S, wherein the heteroaromatic group is optionally substituted with 1, 2 or 3 groups independently selected from
      alkyl,
      haloalkyl,
      cycloalkyl,
      —NR¹⁰R¹⁰,
      halogen,
      alkoxy, which is optionally substituted with —NR¹⁰R¹⁰,
      —CN,
      alkenyl,
      R¹⁰R¹⁰N—CO—,
      —(NR¹⁰═)S(═O)-alkyl,
      cycloalkyl-NR¹⁰—,
      alkyl-NR¹⁰—, wherein the alkyl group is substituted with hydroxy or alkoxy, alkylsulfanyl, and
      a non-aromatic heterocyclic 4-, 5- or 6-membered monocyclic group having 1 or 2 heteroatoms independently selected from 0 and N, which heterocyclic group is optionally substituted with alkyl, hydroxyalkyl or hydroxyl.
11. A composition of embodiment 10, wherein R⁵ is selected from
   (a) phenyl which is substituted with 1, 2 or 3 groups independently selected from
      halogen,
      alkyl,
      alkoxy,
      alkoxy wherein the alkyl group is substituted with 1, 2 or 3 hydroxy groups, hydroxy,
      —CO₂R¹⁰,
      —SF₅,
      1H- or 2H-tetrazolyl; and
   (b) a heteroaromatic 5- or 6-membered monocyclic group having 1, or 2 heteroatoms independently selected from O, N and S, wherein the heteroaromatic group is optionally substituted with 1, 2 or 3 groups independently selected from
      alkyl,
      haloalkyl,
      cycloalkyl,
      —NR¹⁰R¹⁰,
      halogen,
      alkoxy,
      R¹⁰R¹⁰N—CO—,
      —(NR¹⁰═)S(═O)-alkyl,
      cycloalkyl-NR¹⁰—,
      alkyl-NR¹⁰—, wherein the alkyl group is substituted with hydroxy or alkoxy, alkylsulfanyl,
      a non-aromatic heterocyclic 4-, 5- or 6-membered monocyclic group having 1 or 2 heteroatoms independently selected from O and N, which heterocyclic group is optionally substituted with alkyl, hydroxyalkyl or hydroxy.
12. A composition of any one of embodiments 9 to 11, wherein component a) comprises a compound of formula (I), wherein R⁵ is a heteroaromatic 5- or 6-membered monocyclic group having 1 or 2 heteroatoms independently selected from O, N and S, wherein the heteroaromatic group is optionally substituted with 1, 2 or 3 groups as defined in embodiments 9 to 11; or a pharmaceutically acceptable salt, solvate or optical isomer thereof.
13. A composition of embodiment 12, wherein the heteroaromatic group is selected from pyridyl, pyrimidinyl, pyridazinyl and pyrazinyl and is optionally substituted with 1, 2 or 3 groups as defined embodiments 9 to 11.
14. A composition of embodiment 13, wherein the heteroaromatic group is pyrimidinyl which is optionally substituted with 1 or 2 groups as defined in embodiments 9 to 11.
15. A composition of embodiment 14, wherein the heteroaromatic group is pyrimidinyl which is substituted with cycloalkyl.
16. A composition of embodiment 15, wherein the heteroaromatic group is pyrimidinyl which is substituted with cyclopropyl.
17. A composition of any one of embodiments 13 to 16, wherein the heteroaromatic group is pyrimidinyl which is substituted in 2-position.
18. A composition of any one of embodiments 1 to 7, wherein component a) comprises a compound of formula (I), wherein X is N; or a pharmaceutically acceptable salt, solvate or optical isomer thereof.
19. A composition of embodiment 18, wherein R⁵ is selected from
   (a) phenyl which is substituted with 1, 2 or 3 groups independently selected from
      halogen,
      alkyl,
      haloalkyl,
      alkoxy,
      hydroxy,
      —SO₂NR¹⁰R¹⁰,
      —CO₂R¹⁰,
      —CONR¹⁰R¹⁰
      1H- or 2H-tetrazolyl,
      —COO—CH₂CH₂—CH(NH₂)—COOR¹⁰,
      —OCH₂O— (methylenedioxy attached in neighboring positions to the phenyl ring),
      —OCH₂CH₂O— (ethylenedioxy attached in neighboring positions to the phenyl ring),
   (b) a heteroaromatic 5- or 6-membered monocyclic group having 1 or 2 heteroatoms independently selected from O, N and S, wherein the heteroaromatic group is optionally substituted with 1, 2 or 3 groups independently selected from
      alkyl,
      haloalkyl,
      cycloalkyl,
      halogen,
      alkoxy,
      hydroxy,
      —CN,
      alkylsulfanyl,
      —CO₂R¹⁰, and
      1H- or 2H-tetrazole.
20. A composition of embodiment 19, wherein R⁵ is selected from
   (a) phenyl which is substituted with 1 or 2 groups independently selected from
      halogen,
      haloalkyl,
      alkyl, and
      alkoxy,
   (b) a heteroaromatic 5- or 6-membered monocyclic group having 1 or 2 N-heteroatoms, wherein the heteroaromatic group is optionally substituted with 1 or 2 groups independently selected from
      alkyl,
      cycloalkyl,
      halogen,
      alkoxy,
      hydroxy,
      —CN,
      alkylsulfanyl,
      —CO₂R¹⁰, and
      1H- or 2H-tetrazole.
21. A composition of embodiment 19 or 20, wherein the heteroaromatic group is selected from pyridyl, pyrimidinyl, pyridazinyl and pyrazinyl and is optionally substituted with 1, 2 or 3 groups as defined in embodiments 19 or 20.
22. A composition of embodiment 21, wherein the heteroaromatic group is pyridyl or pyrimidinyl which is optionally substituted with 1 or 2 groups as defined in embodiments 19 or 20.
23. A composition of embodiment 22, wherein the heteroaromatic group is pyrimidinyl which is substituted with cycloalkyl.
24. A composition of embodiment 23, wherein the heteroaromatic group is pyrimidinyl which is substituted with cyclopropyl.
25. A composition of embodiments 23 or 24, wherein the heteroaromatic group is pyrimidinyl which is substituted in 2-position.
26. A composition of embodiment 22, wherein the heteroaromatic group is pyridyl which is unsubstituted.
27. A composition of any one of the preceding embodiments, wherein component a) comprises a compound of formula I selected from

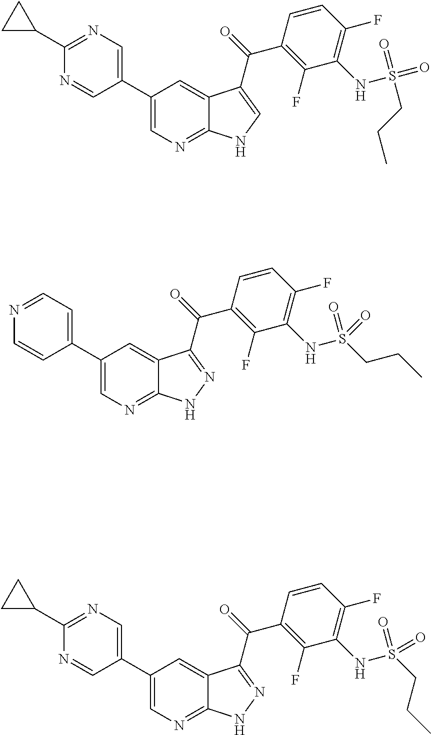 [SMILES: CCCS(=O)(=O)Nc1c(F)ccc(C(=O)c2c[nH]c3ncc(-c4cnc(C5CC5)nc4)cc23)c1F ; CCCS(=O)(=O)Nc1c(F)ccc(C(=O)c2n[nH]c3ncc(-c4ccncc4)cc23)c1F ; CCCS(=O)(=O)Nc1c(F)ccc(C(=O)c2n[nH]c3ncc(-c4cnc(C5CC5)nc4)cc23)c1F]

or a pharmaceutically acceptable salt, solvate or optical isomer thereof.
28. A composition of any one of embodiments 1 to 27, wherein component b) comprises an ERK inhibitor selected from a compound of formula

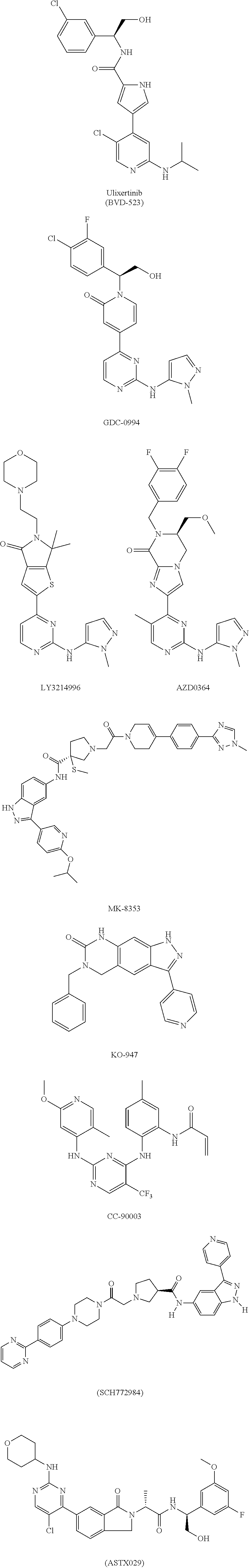

or a pharmaceutically acceptable salt, solvate or optical isomer thereof.
29. A composition of any one of embodiments 1 to 27, wherein component b) comprises a MEK inhibitor selected from a compound of formula

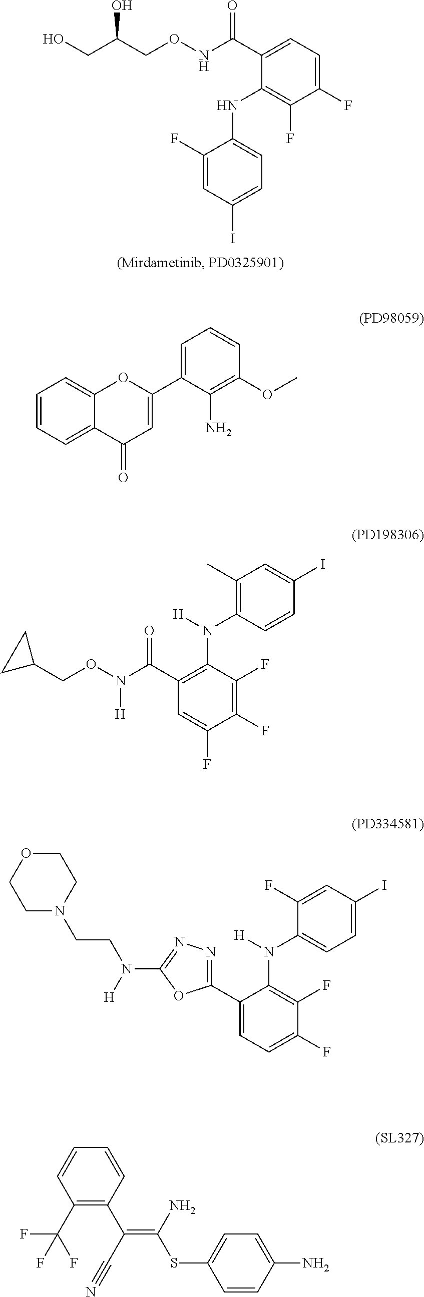 [SMILES: O=C(NOC[C@H](O)CO)c1ccc(F)c(F)c1Nc1ccc(I)cc1F ; COc1cccc(-c2cc(=O)c3ccccc3o2)c1N ; Cc1cc(I)ccc1Nc1c(C(=O)NOCC2CC2)cc(F)c(F)c1F ; Fc1cc(I)ccc1Nc1c(-c2nnc(NCCN3CCOCC3)o2)ccc(F)c1F ; N#C/C(=C(/N)Sc1ccc(N)cc1)c1ccccc1C(F)(F)F]

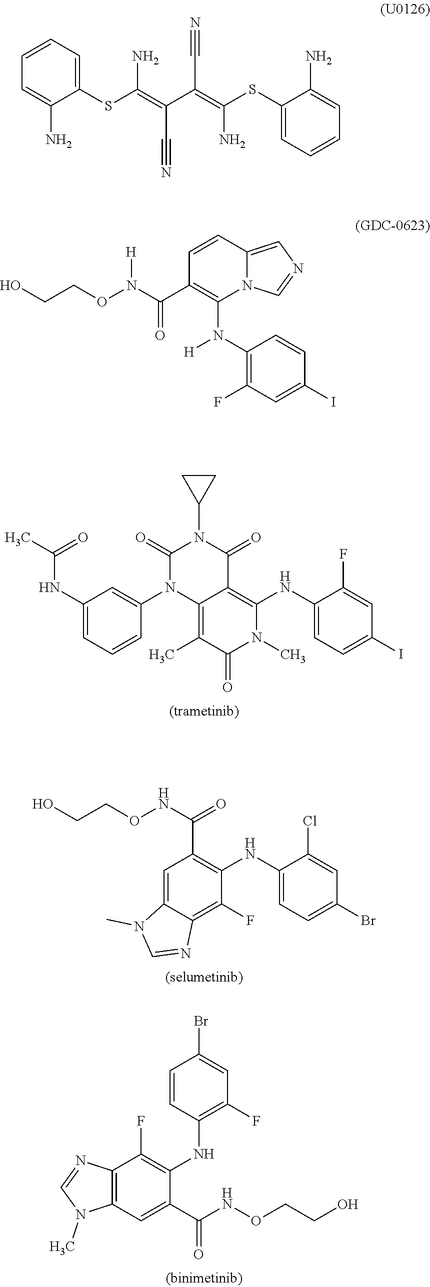 [SMILES: N#CC(=C(/N)Sc1ccccc1N)/C(C#N)=C(\N)Sc1ccccc1N ; O=C(NOCCO)c1ccc2cncn2c1Nc1ccc(I)cc1F ; CC(=O)Nc1cccc(-n2c(=O)n(C3CC3)c(=O)c3c(Nc4ccc(I)cc4F)n(C)c(=O)c(C)c32)c1 ; Cn1cnc2c(F)c(Nc3ccc(Br)cc3Cl)c(C(=O)NOCCO)cc21 ; Cn1cnc2c(F)c(Nc3ccc(Br)cc3F)c(C(=O)NOCCO)cc21]

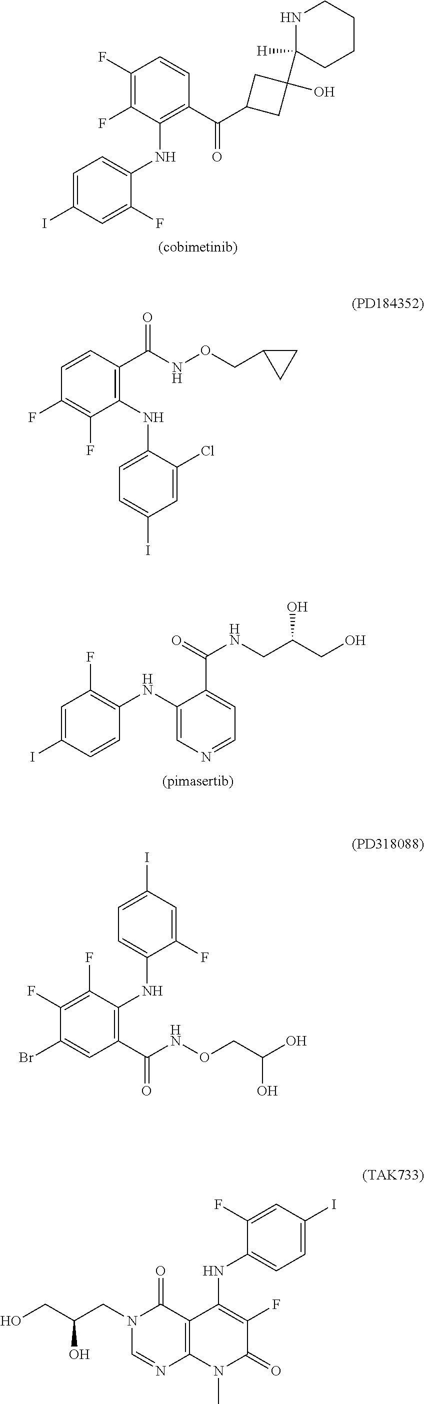 [SMILES: O=C(c1ccc(F)c(F)c1Nc1ccc(I)cc1F)C1CC(O)([C@@H]2CCCCN2)C1 ; O=C(NOCC1CC1)c1ccc(F)c(F)c1Nc1ccc(I)cc1Cl ; O=C(NC[C@H](O)CO)c1ccncc1Nc1ccc(I)cc1F ; O=C(NOCC(O)O)c1cc(Br)c(F)c(F)c1Nc1ccc(I)cc1F ; Cn1c(=O)c(F)c(Nc2ccc(I)cc2F)c2c(=O)n(C[C@@H](O)CO)cnc21]

or a pharmaceutically acceptable salt, solvate or optical isomer thereof.
30. A composition of any one of embodiments 1 to 27, wherein component b) comprises a KRAS inhibitor of the formula

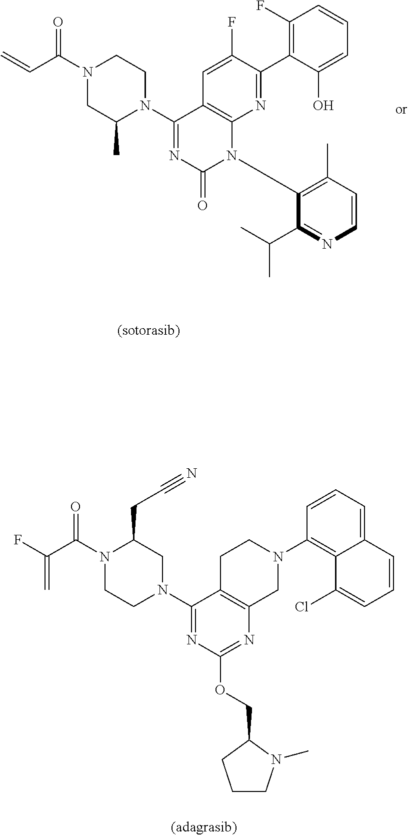

or JNJ-74699157 (ARS-3248), GDC-6036, D-1553, orJDQ433,
or a pharmaceutically acceptable salt, solvate or optical isomer thereof.
31. A composition of any one of embodiments 1 to 27, wherein component b) comprises a SHP2 (PHPS1) inhibitor selected from a compound of formula

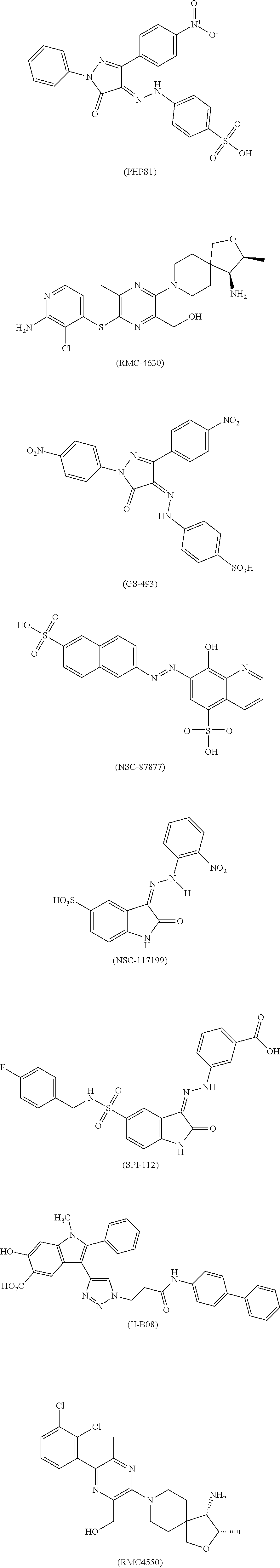

or a pharmaceutically acceptable salt, solvate or optical isomer thereof.
32. A composition of any one of embodiments 1 to 27 or 29, wherein component b) comprises a compound selected from

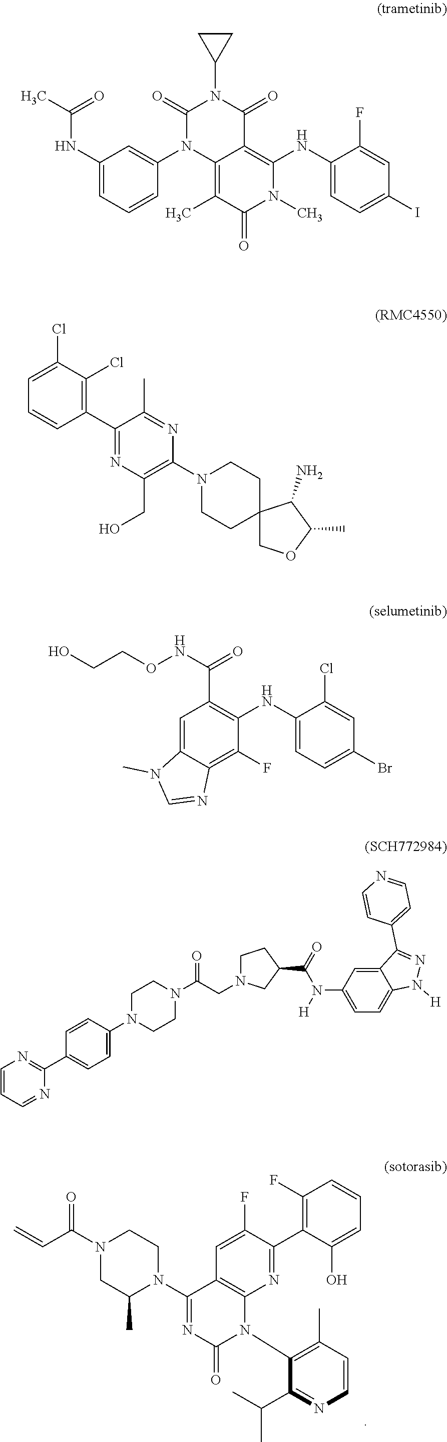

or a pharmaceutically acceptable salt, solvate or optical isomer thereof.
33. A composition of embodiment 1, wherein component a) comprises N-(3-(5-(2-cyclopropyl-pyrimidin-5-yl)-1H-pyrrolo[2,3-b]pyridine-3-carbonyl)-2,6-difluorophenyl)-propane-1-sulfonamide (compound A), or a pharmaceutically acceptable salt, solvate or optical isomer thereof; and component b) comprises a compound selected from trametinib, SCH772984, selumetinib, sotorasib and RMC4550; or a pharmaceutically acceptable salt, solvate or optical isomer thereof.
34. A composition of embodiment 1, wherein component a) comprises N-(2,6-difluoro-3-(5-(pyridin-4-yl)-1H-pyrazolo[3,4-b]pyridine-3-carbonyl)phenyl)propane-1-sulfonamide, or a pharmaceutically acceptable salt, solvate or optical isomer thereof; and component b) comprises a compound of the formula

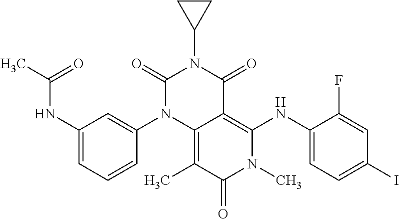 [SMILES: CC(=O)Nc1cccc(-n2c(=O)n(C3CC3)c(=O)c3c(Nc4ccc(I)cc4F)n(C)c(=O)c(C)c32)c1]

or a pharmaceutically acceptable salt, solvate or optical isomer thereof.
35. A composition of embodiment 1, wherein component a) comprises N-(3-(5-(2-cyclopropylpyrimidin-5-yl)-1H-pyrazolo[3,4-b]pyridine-3-carbonyl)-2,6-difluorophenyl)propane-1-sulfonamide, or a pharmaceutically acceptable salt, solvate or optical isomer thereof; and component b) comprises a compound of the formula

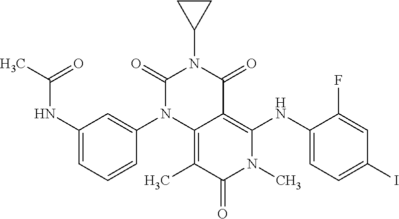 [SMILES: CC(=O)Nc1cccc(-n2c(=O)n(C3CC3)c(=O)c3c(Nc4ccc(I)cc4F)n(C)c(=O)c(C)c32)c1]

or a pharmaceutically acceptable salt, solvate or optical isomer thereof.
36. A combination of components a) and b), wherein component a) comprises at least one compound of formula I as defined in any one of the preceding embodiments and component b) comprises at least one inhibitor as defined in any one of the preceding embodiments.

The term “a compound of the invention” or “a compound of formula I” as used herein means a compound of formula I or a pharmaceutically acceptable salt, solvate or optical isomer thereof.

The invention also relates to a compound of formula I for use in treating cancer. In an embodiment, the cancer is colon cancer or lung cancer, preferably colon or lung cancer and in particular KRAS-mutant lung cancer or KRAS mutant colon cancer. KRAS mutant may be KRAS^{G12C} or KRAS^{G13D}.

Further, the invention also relates to the use of a compound of formula I for preparing a pharmaceutical composition for treating cancer. In an embodiment, the cancer is colon cancer or lung cancer, preferably colon or lung cancer and in particular KRAS-mutant lung cancer or KRAS mutant colon cancer. KRAS mutant may be KRAS^{G12C} or KRAS^{G13D}.

Further, the invention also relates to the use of a compound of formula I for treating cancer. In an embodiment, the cancer is colon cancer or lung cancer, preferably colon or lung cancer and in particular KRAS-mutant lung cancer or KRAS mutant colon cancer. KRAS mutant may be KRAS^{G12C} or KRAS^{G13D}.

In an embodiment, the invention also relates to said compounds of formula I for use in treating cancer as defined above in combination with at least one inhibitor selected from a MEK inhibitor, an ERK inhibitor, and a KRAS, in particular a KRAS^{G12C} inhibitor.

Further, the invention also relates to said pharmaceutical composition or said combination for use in treating cancer. In an embodiment, the cancer is colon cancer or lung cancer, and in particular KRAS-mutant lung cancer or KRAS mutant colon cancer.

Component a) and component b) include the pharmaceutically acceptable salts of the compounds of formula I. The pharmaceutically acceptable salts are especially acid or base addition salts with pharmaceutically acceptable acids or bases. Examples of suitable pharmaceutically acceptable organic and inorganic acids are hydrochloric acid, hydrobromic acid, phosphoric acid, sulfuric acid, sulfamic acid, C₁-C₄-alkylsulfonic acids, such as methanesulfonic acid, cycloaliphatic sulfonic acids, such as S-(+)-10-camphor sulfonic acid, aromatic sulfonic acids, such as benzenesulfonic acid and toluenesulfonic acid, di- and tricarboxylic acids and hydroxycarboxylic acids having 2 to 10 carbon atoms, such as oxalic acid, malonic acid, maleic acid, fumaric acid, lactic acid, tartaric acid, citric acid, glycolic acid, adipic acid and benzoic acid. Other utilizable acids are described, e.g., in Fortschritte der Arzneimittelforschung [Advances in drug research], Volume 10, pages 224 ff., Birkhäuser Verlag, Basel and Stuttgart, 1966. Examples of suitable pharmaceutically acceptable organic and inorganic bases are alkali metal hydroxides, such as sodium hydroxide or potassium hydroxide, alkaline earth metal hydroxides such as calcium or magnesium hydroxide, ammonium hydroxide, organic nitrogen bases such as dimethylamine, trimethylamine, ethanolamine, diethanolamine, triethanolamine, choline, 2-amino-2-hydroxymethyl-propane-1,3-diol, meglumine, procaine etc. L-arginine, L-lysine, ethylenediamine, or hydroxyethylpyrrolidine.

The invention also includes any tautomeric, crystal and polymorphic form of said compounds and salts and mixtures thereof.

The invention also includes solvates such as hydrates.

The compounds contemplated may contain one or more chiral centers, and exist in different optically active forms such enantiomers and diastereomers.

The organic moieties mentioned in the above definitions of the variables are—like the term halogen—collective terms for individual listings of the individual group members. The prefix Cₙ-Cₘ indicates in each case the possible number of carbon atoms in the group.

The term halogen denotes in each case fluorine, bromine, chlorine or iodine, in particular fluorine or chlorine, and most preferably fluorine.

Alkyl is a straight-chain or branched alkyl group which is preferably a C₁-C₆-alkyl group, i.e. an alkyl group having from 1 to 6 carbon atoms, and more preferably a C₁-C₄-alkyl group. Examples of an alkyl group are methyl, ethyl, n-propyl, iso-propyl, n-butyl, 2-butyl, iso-butyl, tert-butyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl.

The definition of alkyl is likewise applicable to any group which includes an alkyl group.

Haloalkyl is a halogenated alkyl group as defined above, wherein at least one, e.g. 1, 2, 3, 4 or all of the hydrogen atoms are replaced by 1, 2, 3, 4 or a corresponding number of identical or different halogen atoms, such as trifluoromethyl, chloromethyl, bromomethyl, difluoromethyl, fluoromethyl, difluoroethyl, etc. Particular examples include the fluorinated C₁-C₄ alkyl groups as defined, such as trifluoromethyl, difluoromethyl, fluoromethyl, difluoroethyl, 2,2,2-trifluoroethyl or 3,3,3-trifluoropropyl.

Cycloalkyl is a cycloaliphatic radical which is preferably C₃-C₃-cycloalkyl, i.e. a cycloalkyl group having from 3 to 8 carbon atoms. In particular, 3 to 6 carbon atoms form the cyclic structure, such as cyclopropyl, cyclobutyl, cyclopentyl and cyclohexyl. The cyclic structure may be unsubstituted or may carry 1, 2, 3 or 4 C₁-C₄ alkyl radicals, preferably one or more methyl radicals.

Carbonyl is >C═O.

Aminocarbonyl is NH₂C(O)—.

Alkenyl is a singly unsaturated hydrocarbon radical which is preferably a C₂-C₆-alkenyl group, i.e. an alkenyl group having 2, 3, 4, 5 or 6 carbon atoms, e.g. vinyl, allyl (2-propen-1-yl), 1-propen-1-yl, 2-propen-2-yl, methallyl(2-methylprop-2-en-1-yl) and the like. C₃-C₅-Alkenyl is, in particular, allyl, 1-methylprop-2-en-1-yl, 2-buten-1-yl, 3-buten-1-yl, methallyl, 2-penten-1-yl, 3-penten-1-yl, 4-penten-1-yl, 1-methylbut-2-en-1-yl or 2-ethylprop-2-en-1-yl, 2-hexen-1-yl.

Alkinyl is a singly unsaturated hydrocarbon radical which is preferably a C₂-C₆-alkinyl group, i.e. an alkinyl group having 2, 3, 4, 5 or 6 carbon atoms, e.g. ethynyl, 2-propyn-1-yl, 1-propyn-1-yl, 2-propyn-2-yl and the like. C₃-C₅-Alkinyl is, in particular, 2-propyn-1-yl, 2-butyn-1-yl, 3-butyn-1-yl, 2-pentyn-1-yl, 3-pentyn-1-yl, 4-pentyn-1-yl.

A heteroaromatic (or heteroaryl) group is a 5- or 6-membered monocyclic aromatic group having 1, 2 or 3, preferably 1 or 2, heteroatoms selected from O, N and S. The heteroaryl or heteroaromatic group may be bound to the neighboring group via a carbon atom (C-bound) or via a nitrogen heteroatom (N-bound). Preferred heteroaromatic radicals comprise 1 nitrogen atom as ring member atom and optionally 1 or 2 further heteroatoms as ring members, which are selected, independently of each other from O, S and N. Examples are:
C-bound, 5-membered, heteroaromatic rings:
2-furyl, 3-furyl, 5-furyl, 2-thienyl, 3-thienyl, 5-thienyl, pyrrol-2-yl, pyrrol-3-yl, pyrrol-5-yl, pyrazol-3-yl, pyrazol-4-yl, pyrazol-5-yl, isoxazol-3-yl, isoxazol-4-yl, isoxazol-5-yl, isothiazol-3-yl, isothiazol-4-yl, isothiazol-5-yl, imidazol-2-yl, imidazol-4-yl, imidazol-5-yl, oxazol-2-yl, oxazol-4-yl, oxazol-5-yl, thiazol-2-yl, thiazol-4-yl, thiazol-5-yl, 1,2,3-oxadiazol-imidazol-4-yl,4-yl, 1,2,3-oxadiazol-5-yl, 1,2,4-oxadiazol-3-yl, 1,2,4,-oxadiazol-5-yl, 1,3,4-oxadiazol-2-yl, 1,2,3-thiadiazol-4-yl, 1,2,3-thiadiazol-5-yl, 1,2,4-thiadiazol-3-yl, 1,2,4-thiadiazol-5-yl, 1,3,4-thiadiazolyl-2-yl, 1,2,3-triazol-4-yl, 1,2,4-triazol-3-yl, tetrazol-5-yl;
C-bound, 6-membered, heteroaromatic rings:
pyridin-2-yl, pyridin-3-yl (3-pyridyl), pyridin-4-yl (4-pyridyl), pyridin-5-yl, pyridazin-3-yl, pyridazin-4-yl, pyridazin-6-yl, pyrimidin-2-yl, pyrimidin-4-yl, pyrimidin-5-yl, pyrazin-2-yl, pyrazin-5-yl, 1,3,5-triazin-2-yl, 1,2,4-triazin-3-yl, 1,2,4-triazin-5-yl, 1,2,4-triazin-6-yl, 1,2,4,5-tetrazin-3-yl;
N-bound, 5-membered, heteroaromatic rings:
pyrrol-1-yl, pyrazol-1-yl, imidazol-1-yl, 1,2,3-triazol-1-yl, 1,2,4-triazol-1-yl.

The heteroaryl or heteroaromatic group may also be fused with a phenyl group. Examples are quinolinyl, isoquinolinyl, indolyl, indolizinyl, isoindolyl, 4-, 5-, 6- or 7-azaindole, indazolyl, benzofuryl, benzthienyl, benzo[b]thiazolyl, benzoxazolyl, benzthiazolyl, benzimidazolyl, imidazo[b]thiazolyl, thieno[b]pyridyl, imidazo[a]pyridyl, pyrazo[a]pyridyl and pyrrol[d]pyrimidyl.

A non-aromatic 5- or 6-membered group (heterocyclic group) may be saturated or partially unsaturated, preferably saturated, and includes 1, 2 or 3, preferably 1 or 2, heteroatoms selected from O, N and S. The heterocyclic radicals may be bound via a carbon atom (C-bound) or a nitrogen atom (N-bound). Preferred heterocyclic groups comprise 1 nitrogen atom as ring member atom and optionally 1 or 2 further heteroatoms as ring members, which are selected, independently of each other from O, S and N. Examples are:
C-bound, 4-membered, saturated rings, such as
azetidin-2-yl, azetidin-3-yl, oxetan-2-yl, oxetan-3-yl;
C-bound, 5-membered, saturated rings, such as
tetrahydrofuran-2-yl, tetrahydrofuran-3-yl, tetrahydrothien-2-yl, tetrahydrothien-3-yl, tetrahydropyrrol-2-yl, tetrahydropyrrol-3-yl, tetrahydropyrazol-3-yl, tetrahydro-pyrazol-4-yl, tetrahydroisoxazol-3-yl, tetrahydroisoxazol-4-yl, tetrahydroisoxazol-5-yl, 1,2-oxathiolan-3-yl, 1,2-oxathiolan-4-yl, 1,2-oxathiolan-5-yl, tetrahydroisothiazol-3-yl, tetrahydroisothiazol-4-yl, tetrahydroisothiazol-5-yl, 1,2-dithiolan-3-yl, 1,2-dithiolan-4-yl, tetrahydroimidazol-2-yl, tetrahydroimidazol-4-yl, tetrahydrooxazol-2-yl, tetrahydrooxazol-4-yl, tetrahydrooxazol-5-yl, tetrahydrothiazol-2-yl, tetrahydrothiazol-4-yl, tetrahydrothiazol-5-yl, 1,3-dioxolan-2-yl, 1,3-dioxolan-4-yl, 1,3-oxathiolan-2-yl, 1,3-oxathiolan-4-yl, 1,3-oxathiolan-5-yl, 1,3-dithiolan-2-yl, 1,3-dithiolan-4-yl, 1,3,2-dioxathiolan-4-yl;
C-bound, 6-membered, saturated rings, such as
tetrahydropyran-2-yl, tetrahydropyran-3-yl, tetrahydropyran-4-yl, piperidin-2-yl, piperidin-3-yl, piperidin-4-yl, tetrahydrothiopyran-2-yl, tetrahydrothiopyran-3-yl, tetrahydrothiopyran-4-yl, 1,3-dioxan-2-yl, 1,3-dioxan-4-yl, 1,3-dioxan-5-yl, 1,4-dioxan-2-yl, 1,3-dithian-2-yl, 1,3-dithian-4-yl, 1,3-dithian-5-yl, 1,4-dithian-2-yl, 1,3-oxathian-2-yl, 1,3-oxathian-4-yl, 1,3-oxathian-5-yl, 1,3-oxathian-6-yl, 1,4-oxathian-2-yl, 1,4-oxathian-3-yl, 1,2-dithian-3-yl, 1,2-dithian-4-yl, hexahydropyrimidin-2-yl, hexahydropyrimidin-4-yl, hexahydropyrimidin-5-yl, hexahydropyrazin-2-yl, hexahydropyridazin-3-yl, hexahydropyridazin-4-yl, tetrahydro-1,3-oxazin-2-yl, tetrahydro-1,3-oxazin-4-yl, tetrahydro-1,3-oxazin-5-yl, tetrahydro-1,3-oxazin-6-yl, tetrahydro-1,3-thiazin-2-yl, tetrahydro-1,3-thiazin-4-yl, tetrahydro-1,3-thiazin-5-yl, tetrahydro-1,3-thiazin-6-yl, tetrahydro-1,4-thiazin-2-yl, tetrahydro-1,4-thiazin-3-yl, tetrahydro-1,4-oxazin-2-yl, tetrahydro-1,4-oxazin-3-yl, tetrahydro-1,2-oxazin-3-yl, tetrahydro-1,2-oxazin-4-yl, tetrahydro-1,2-oxazin-5-yl, tetrahydro-1,2-oxazin-6-yl;
N-bound, 4-membered, saturated rings, such as
azetidin-1-yl;
N-bound, 5-membered, saturated rings, such as
tetrahydropyrrol-1-yl (pyrrolidin-1-yl), tetrahydropyrazol-1-yl, tetrahydroisoxazol-2-yl, tetrahydroisothiazol-2-yl, tetrahydroimidazol-1-yl, tetrahydrooxazol-3-yl, tetrahydrothiazol-3-yl;
N-bound, 6-membered, saturated rings, such as
piperidin-1-yl, hexahydropyrimidin-1-yl, hexahydropyrazin-1-yl (piperazin-1-yl), hexahydro-pyridazin-1-yl, tetrahydro-1,3-oxazin-3-yl, tetrahydro-1,3-thiazin-3-yl, tetrahydro-1,4-thiazin-4-yl, tetrahydro-1,4-oxazin-4-yl (morpholin-1-yl), tetrahydro-1,2-oxazin-2-yl;
C-bound, 5-membered, partially unsaturated rings, such as
2,3-dihydrofuran-2-yl, 2,3-dihydrofuran-3-yl, 2,5-dihydrofuran-2-yl, 2,5-di-hydrofuran-3-yl, 4,5-dihydrofuran-2-yl, 4,5-dihydrofuran-3-yl, 2,3-dihydro-thien-2-yl, 2,3-dihydrothien-3-yl, 2,5-dihydrothien-2-yl, 2,5-dihydrothien-3-yl, 4,5-dihydrothien-2-yl, 4,5-dihydrothien-3-yl, 2,3-dihydro-1H-pyrrol-2-yl, 2,3-dihydro-1H-pyrrol-3-yl, 2,5-dihydro-1H-pyrrol-2-yl, 2,5-dihydro-1H-pyrrol-3-yl, 4,5-dihydro-1H-pyrrol-2-yl, 4,5-dihydro-1H-pyrrol-3-yl, 3,4-dihydro-2H-pyrrol-2-yl, 3,4-dihydro-2H-pyrrol-3-yl, 3,4-dihydro-5H-pyrrol-2-yl, 3,4-dihydro-5H-pyrrol-3-yl, 4,5-dihydro-1H-pyrazol-3-yl, 4,5-dihydro-1H-pyrazol-4-yl, 4,5-dihydro-1H-pyrazol-5-yl, 2,5-dihydro-1H-pyrazol-3-yl, 2,5-dihydro-1H-pyrazol-4-yl, 2,5-dihydro-1H-pyrazol-5-yl, 4,5-dihydroisoxazol-3-yl, 4,5-dihydroisoxazol-4-yl, 4,5-dihydroisoxazol-5-yl, 2,5-dihydroisoxazol-3-yl, 2,5-dihydroisoxazol-4-yl, 2,5-dihydroisoxazol-5-yl, 2,3-dihydroisoxazol-3-yl, 2,3-dihydroisoxazol-4-yl, 2,3-dihydroisoxazol-5-yl, 4,5-dihydroisothiazol-3-yl, 4,5-dihydroisothiazol-4-yl, 4,5-dihydroisothiazol-5-yl, 2,5-dihydroisothiazol-3-yl, 2,5-dihydroisothiazol-4-yl, 2,5-dihydroisothiazol-5-yl, 2,3-dihydroisothiazol-3-yl, 2,3-dihydroisothiazol-4-yl, 2,3-dihydroisothiazol-5-yl, 4,5-dihydro-1H-imidazol-2-yl, 4,5-dihydro-1H-imidazol-4-yl, 4,5-dihydro-1H-imidazol-5-yl, 2,5-dihydro-1H-imidazol-2-yl, 2,5-dihydro-1H-imidazol-4-yl, 2,5-dihydro-1H-imidazol-5-yl, 2,3-dihydro-1H-imidazol-2-yl, 2,3-dihydro-1H-imidazol-4-yl, 4,5-dihydro-oxazol-2-yl, 4,5-dihydrooxazol-4-yl, 4,5-dihydrooxazol-5-yl, 2,5-dihydrooxazol-2-yl, 2,5-dihydrooxazol-4-yl, 2,5-dihydrooxazol-5-yl, 2,3-dihydrooxazol-2-yl, 2,3-dihydrooxazol-4-yl, 2,3-dihydrooxazol-5-yl, 4,5-dihydrothiazol-2-yl, 4,5-dihydrothiazol-4-yl, 4,5-dihydrothiazol-5-yl, 2,5-dihydrothiazol-2-yl, 2,5-dihydrothiazol-4-yl, 2,5-dihydrothiazol-5-yl, 2,3-dihydrothiazol-2-yl, 2,3-dihydrothiazol-4-yl, 2,3-dihydrothiazol-5-yl, 1,3-dioxol-2-yl, 1,3-dioxol-4-yl, 1,3-dithiol-2-yl, 1,3-dithiol-4-yl, 1,3-oxathiol-2-yl, 1,3-oxathiol-4-yl, 1,3-oxathiol-5-yl;
C-bound, 6-membered, partially unsaturated rings, such as
2H-3,4-dihydropyran-6-yl, 2H-3,4-dihydropyran-5-yl, 2H-3,4-dihydropyran-4-yl, 2H-3,4-dihydropyran-3-yl, 2H-3,4-dihydropyran-2-yl, 2H-3,4-dihydrothiopyran-6-yl, 2H-3,4-dihydrothiopyran-5-yl, 2H-3,4-dihydrothiopyran-4-yl, 2H-3,4-dihydrothiopyran-3-yl, 2H-3,4-dihydrothiopyran-2-yl, 1,2,3,4-tetrahydropyridin-6-yl, 1,2,3,4-tetrahydropyridin-5-yl, 1,2,3,4-tetrahydropyridin-4-yl, 1,2,3,4-tetra-hydropyridin-3-yl, 1,2,3,4-tetrahydropyridin-2-yl, 2H-5,6-dihydropyran-2-yl, 2H-5,6-dihydropyran-3-yl, 2H-5,6-dihydropyran-4-yl, 2H-5,6-dihydropyran-5-yl, 2H-5,6-dihydropyran-6-yl, 2H-5,6-dihydrothiopyran-2-yl, 2H-5,6-dihydrothiopyran-3-yl, 2H-5,6-dihydrothiopyran-4-yl, 2H-5,6-dihydrothiopyran-5-yl, 2H-5,6-dihydrothiopyran-6-yl, 1,2,5,6-tetrahydropyridin-2-yl, 1,2,5,6-tetrahydropyridin-3-yl, 1,2,5,6-tetrahydropyridin-4-yl, 1,2,5,6-tetrahydropyridin-5-yl, 1,2,5,6-tetrahydropyridin-6-yl, 2,3,4,5-tetrahydropyridin-2-yl, 2,3,4,5-tetrahydropyridin-3-yl, 2,3,4,5-tetrahydropyridin-4-yl, 2,3,4,5-tetrahydropyridin-5-yl, 2,3,4,5-tetrahydropyridin-6-yl, 4H-pyran-2-yl, 4H-pyran-3-yl-, 4H-pyran-4-yl, 4H-thiopyran-2-yl, 4H-thiopyran-3-yl, 4H-thiopyran-4-yl, 1,4-dihydropyridin-2-yl, 1,4-dihydropyridin-3-yl, 1,4-dihydropyridin-4-yl, 2H-pyran-2-yl, 2H-pyran-3-yl, 2H-pyran-4-yl, 2H-pyran-5-yl, 2H-pyran-6-yl, 2H-thiopyran-2-yl, 2H-thiopyran-3-yl, 2H-thiopyran-4-yl, 2H-thiopyran-5-yl, 2H-thiopyran-6-yl, 1,2-dihydropyridin-2-yl, 1,2-dihydro-pyridin-3-yl, 1,2-dihydropyridin-4-yl, 1,2-dihydropyridin-5-yl, 1,2-dihydro-pyridin-6-yl, 3,4-dihydropyridin-2-yl, 3,4-dihydropyridin-3-yl, 3,4-dihydro-pyridin-4-yl, 3,4-dihydropyridin-5-yl, 3,4-dihydropyridin-6-yl, 2,5-dihydropyridin-2-yl, 2,5-dihydropyridin-3-yl, 2,5-dihydropyridin-4-yl, 2,5-dihydropyridin-5-yl, 2,5-dihydropyridin-6-yl, 2,3-dihydropyridin-2-yl, 2,3-dihydropyridin-3-yl, 2,3-dihydropyridin-4-yl, 2,3-dihydro-pyridin-5-yl, 2,3-dihydropyridin-6-yl, 2H-5,6-dihydro-1,2-oxazin-3-yl, 2H-5,6-dihydro-1,2-oxazin-4-yl, 2H-5,6-dihydro-1,2-oxazin-5-yl, 2H-5,6-dihydro-1,2-oxazin-6-yl, 2H-5,6-dihydro-1,2-thiazin-3-yl, 2H-5,6-dihydro-1,2-thiazin-4-yl, 2H-5,6-dihydro-1,2-thiazin-5-yl, 2H-5,6-dihydro-1,2-thiazin-6-yl, 4H-5,6-dihydro-1,2-oxazin-3-yl, 4H-5,6-dihydro-1,2-oxazin-4-yl, 4H-5,6-dihydro-1,2-oxazin-5-yl, 4H-5,6-dihydro-1,2-oxazin-6-yl, 4H-5,6-dihydro-1,2-thiazin-3-yl, 4H-5,6-dihydro-1,2-thiazin-4-yl, 4H-5,6-dihydro-1,2-thiazin-5-yl, 4H-5,6-dihydro-1,2-thiazin-6-yl, 2H-3,6-dihydro-1,2-oxazin-3-yl, 2H-3,6-dihydro-1,2-oxazin-4-yl, 2H-3,6-dihydro-1,2-oxazin-5-yl, 2H-3,6-dihydro-1,2-oxazin-6-yl, 2H-3,6-dihydro-1,2-thiazin-3-yl, 2H-3,6-dihydro-1,2-thiazin-4-yl, 2H-3,6-dihydro-1,2-thiazin-5-yl, 2H-3,6-dihydro-1,2-thiazin-6-yl, 2H-3,4-dihydro-1,2-oxazin-3-yl, 2H-3,4-dihydro-1,2-oxazin-4-yl, 2H-3,4-dihydro-1,2-oxazin-5-yl, 2H-3,4-dihydro-1,2-oxazin-6-yl, 2H-3,4-dihydro-1,2-thiazin-3-yl, 2H-3,4-dihydro-1,2-thiazin-4-yl, 2H-3,4-dihydro-1,2-thiazin-5-yl, 2H-3,4-dihydro-1,2-thiazin-6-yl, 2,3,4,5-tetrahydropyridazin-3-yl, 2,3,4,5-tetrahydropyridazin-4-yl, 2,3,4,5-tetrahydropyridazin-5-yl, 2,3,4,5-tetrahydro-pyridazin-6-yl, 3,4,5,6-tetrahydropyridazin-3-yl, 3,4,5,6-tetrahydropyridazin-4-yl, 1,2,5,6-tetrahydropyridazin-3-yl, 1,2,5,6-tetrahydropyridazin-4-yl, 1,2,5,6-tetra-hydropyridazin-5-yl, 1,2,5,6-tetrahydropyridazin-6-yl, 1,2,3,6-tetrahydro-pyridazin-3-yl, 1,2,3,6-tetrahydropyridazin-4-yl, 4H-5,6-dihydro-1,3-oxazin-2-yl, 4H-5,6-dihydro-1,3-oxazin-4-yl, 4H-5,6-dihydro-1,3-oxazin-5-yl, 4H-5,6-dihydro-1,3-oxazin-6-yl, 4H-5,6-dihydro-1,3-thiazin-2-yl, 4H-5,6-dihydro-1,3-thiazin-4-yl, 4H-5,6-dihydro-1,3-thiazin-5-yl, 4H-5,6-dihydro-1,3-thiazin-6-yl, 3,4,5-6-tetrahydropyrimidin-2-yl, 3,4,5,6-tetrahydropyrimidin-4-yl, 3,4,5,6-tetrahydropyrimidin-5-yl, 3,4,5,6-tetrahydropyrimidin-6-yl, 1,2,3,4-tetrahydropyrazin-2-yl, 1,2,3,4-tetrahydropyrazin-5-yl, 1,2,3,4-tetrahydro-pyrimidin-2-yl, 1,2,3,4-tetrahydropyrimidin-4-yl, 1,2,3,4-tetrahydropyrimidin-5-yl, 1,2,3,4-tetrahydropyrimidin-6-yl, 2,3-dihydro-1,4-thiazin-2-yl, 2,3-dihydro-1,4-thiazin-3-yl, 2,3-dihydro-1,4-thiazin-5-yl, 2,3-dihydro-1,4-thiazin-6-yl, 2H-1,3-oxazin-2-yl, 2H-1,3-oxazin-4-yl, 2H-1,3-oxazin-5-yl, 2H-1,3-oxazin-6-yl, 2H-1,3-thiazin-2-yl, 2H-1,3-thiazin-4-yl, 2H-1,3-thiazin-5-yl, 2H-1,3-thiazin-6-yl, 4H-1,3-oxazin-2-yl, 4H-1,3-oxazin-4-yl, 4H-1,3-oxazin-5-yl, 4H-1,3-oxazin-6-yl, 4H-1,3-thiazin-2-yl, 4H-1,3-thiazin-4-yl, 4H-1,3-thiazin-5-yl, 4H-1,3-thiazin-6-yl, 6H-1,3-oxazin-2-yl, 6H-1,3-oxazin-4-yl, 6H-1,3-oxazin-5-yl, 6H-1,3-oxazin-6-yl, 6H-1,3-thiazin-2-yl, 6H-1,3-oxazin-4-yl, 6H-1,3-oxazin-5-yl, 6H-1,3-thiazin-6-yl, 2H-1,4-oxazin-2-yl, 2H-1,4-oxazin-3-yl, 2H-1,4-oxazin-5-yl, 2H-1,4-oxazin-6-yl, 2H-1,4-thiazin-2-yl, 2H-1,4-thiazin-3-yl, 2H-1,4-thiazin-5-yl, 2H-1,4-thiazin-6-yl, 4H-1,4-oxazin-2-yl, 4H-1,4-oxazin-3-yl, 4H-1,4-thiazin-2-yl, 4H-1,4-thiazin-3-yl, 1,4-dihydropyridazin-3-yl, 1,4-dihydropyridazin-4-yl, 1,4-dihydropyridazin-5-yl, 1,4-dihydropyridazin-6-yl, 1,4-dihydropyrazin-2-yl, 1,2-dihydropyrazin-2-yl, 1,2-dihydropyrazin-3-yl, 1,2-dihydropyrazin-5-yl, 1,2-dihydropyrazin-6-yl, 1,4-dihydropyrimidin-2-yl, 1,4-dihydropyrimidin-4-yl, 1,4-dihydropyrimidin-5-yl, 1,4-dihydropyrimidin-6-yl, 3,4-dihydropyrimidin-2-yl, 3,4-dihydropyrimidin-4-yl, 3,4-dihydropyrimidin-5-yl or 3,4-dihydropyrimidin-6-yl;

Any group containing heteroatoms may contain 1, 2 or 3 heteroatoms which may be the same or different.

The compounds of component a) including the pharmaceutically acceptable salts, solvates and stereoisomers thereof, are described and prepared in WO2019149738, WO2020016243, WO2021018820 and WO2021144287. These publications are incorporated herein in their entirety by reference, in particular to the single compounds therein and their preparation. The acid or base addition salts are prepared in a customary manner by mixing the free base with a corresponding acid or by mixing the free acid with the desired base. Optionally, the reaction is carried out in solution in an organic solvent, for example a lower alcohol, such as methanol, ethanol or propanol, an ether, such as methyl tert-butyl ether or diisopropyl ether, a ketone, such as acetone or methyl ethyl ketone, or an ester, such as EtOAc.

The compounds of component b) are well-known in the art and are commercially available. For example, RAS, MEK and ERK inhibitors can be obtained by methods according to the publications disclosed, for example, in WO201720462 or by methods analogous thereto.

An inhibitor need not completely abrogate the biological function of a target protein or polypeptide, and in some embodiments reduces the activity by at least 20%, 30%, 40%, 50%, 60%, 70%, 80%, 90%, 95%, or 99%.

The pharmaceutical composition or combination of the invention is useful for treating cancer, in particular colon and lung cancer. a KRAS mutant colon or lung cancer. They are especially useful for treating a KRAS-mutant lung cancer, KRAS-mutant breast cancer or a KRAS-mutant colon cancer. The cancer may be a cancer that has or has acquired resistance to treatment with a MEK inhibitor, an ERK inhibitor, a KRAS inhibitor and/or a SHP2 inhibitor.

As used herein, the expression “colon cancer” means colorectal cancer (CRC), i.e. bowel cancer, colon cancer, or rectal cancer. The term “KRAS-mutant cancer” is well known to the skilled person. A comprehensive overview of RAS mutations, including KRAS-mutations, in cancer was reported by Prior et al (2012) Cancer Res; 2457-67. KRAS-mutant cells promote oncogenesis due to being mutationally activated, in most cases, at codon 12, 13 and 61. In total forty-four separate point mutations have been characterized in RAS isoforms, with 99.2% in codons 12, 13 and 61. The protein product of the normal KRAS gene performs an essential function in normal tissue signaling, and the mutation of a KRAS gene is an essential step in the development of many cancers.

The pharmaceutical composition or combination of the invention may be administered as a combined preparation for simultaneous, separate or sequential use in the treatment of cancer in a subject. By simultaneous administration is meant a treatment regime wherein the individual components are administered together, either in the form of a single pharmaceutical composition or device comprising or containing both components, or as separate compositions or devices, each comprising one of the components, administered simultaneously. Such combinations of the separate individual components for simultaneous combination may be provided in the form of a kit-of-parts. Administration of more than one drug may occur at the same time, but not necessarily via the same route of administration. For example, one drug may be provided orally whereas the other drug may be provided intravenously during a patients visit to a hospital. “Separate administration” includes the administration of the drugs in separate form and/or at separate moments in time, but again, not necessarily via the same route of administration. “Sequentially” of “sequential administration” indicates that the administration of a first drug if followed, immediately or in time, by the administration of the second drug, but again, not necessarily via the same route of administration.

The pharmaceutical composition or combination of the invention may comprise component a) and component b) in a largely varying ratio depending on the efficacy of the active compounds and the tumor that has to be treated. In general, the ratio of component a) to is in the range of 30:1 to 1:10.

The pharmaceutical composition or combination of the invention optionally comprises an inert carrier (e.g. a pharmaceutically acceptable excipient) and, where appropriate, other drugs. These compositions and combinations can, for example, be administered orally, rectally, transdermally, subcutaneously, intraperitoneally, intravenously, intramuscularly or intranasally.

The pharmaceutical composition or combination of the invention may be provided as solid medicinal forms, such as powders, granules, tablets, in particular film tablets, lozenges, sachets, cachets, sugar-coated tablets, capsules, such as hard gelatin capsules and soft gelatin capsules, or suppositories, and liquid medicinal forms, such as solutions, emulsions, in particular oil-in-water emulsions, suspensions, for example lotions, injection preparations and infusion preparations. In addition, it is also possible to use liposomes or microspheres.

When producing the compositions or combinations, the compounds according to the invention are optionally mixed or diluted with one or more carriers (excipients). Carriers (excipients) can be solid, semisolid or liquid materials which serve as vehicles, carriers or medium for the active compound.

Suitable carriers (excipients) are listed in the specialist medicinal monographs. In addition, the formulations can comprise pharmaceutically acceptable auxiliary substances, such as wetting agents; emulsifying and suspending agents; preservatives; antioxidants; antiirritants; chelating agents; coating auxiliaries; emulsion stabilizers; film formers; gel formers; odor masking agents; taste corrigents; resins; hydrocolloids; solvents; solubilizers; neutralizing agents; diffusion accelerators; pigments; quaternary ammonium compounds; refatting and overfatting agents; raw materials for ointments, creams or oils; silicone derivatives; spreading auxiliaries; stabilizers; sterilants; suppository bases; tablet auxiliaries, such as binders, fillers, glidants, disintegrants or coatings; propellants; drying agents; opacifiers; thickeners; waxes; plasticizers and white mineral oils. A formulation in this regard is based on specialist knowledge as described, for example, in Fiedler, H. P., Lexikon der Hilfsstoffe für Pharmazie, Kosmetik und angrenzende Gebiete [Encyclopedia of auxiliary substances for pharmacy, cosmetics and related fields], 4^{th} edition, Aulendorf: ECV-Editio-Cantor-Verlag, 1996.

The compositions and combinations of the invention may also be combined with other therapeutic agents. The invention therefore further relates to a combination comprising a composition or combination of the invention with one or more further therapeutic agents, in particular for use in treating cancer. Such combination therapies may be administered adjunctively. By adjunctive administration is meant the coterminous or overlapping administration of each of the components in the form of separate pharmaceutical compositions or devices. This regime of therapeutic administration of two or more therapeutic agents is referred to generally by those skilled in the art and herein as adjunctive therapeutic administration; it is also known as add-on therapeutic administration. Any and all treatment regimes in which a patient receives separate but coterminous or overlapping therapeutic administration of the compositions and combinations of the invention and at least one further therapeutic agent are within the scope of the current invention. In one embodiment of adjunctive therapeutic administration as described herein, a patient is typically stabilized on a therapeutic administration of one or more of the components for a period of time and then receives administration of another component.

The combination therapies of the invention may also be administered simultaneously as described above.

Suitable agents for use in combination with the compounds of the inventions include for example:

In an embodiment the invention relates to a method of treating cancer, in particular lung and colon cancer, KRAS mutant lung and Kras mutant colon cancer which comprises administering an effective amount of a composition or combination of the invention to a subject in need thereof.

In an embodiment, the compounds of the invention are administered in a dosage of 10-1000 mg, preferably in a dosage between 100 and 500 mg. The compounds can be administered once or several times a day.

The following compounds or a pharmaceutically acceptable salt, solvate or optical isomer thereof are useful as component a) in the compositions and combinations of the invention.

Their preparation and MKK4 inhibiting activity and selectivity is described in detail in WO2019149738, WO2020016243, WO2021018820 and WO2021144287:

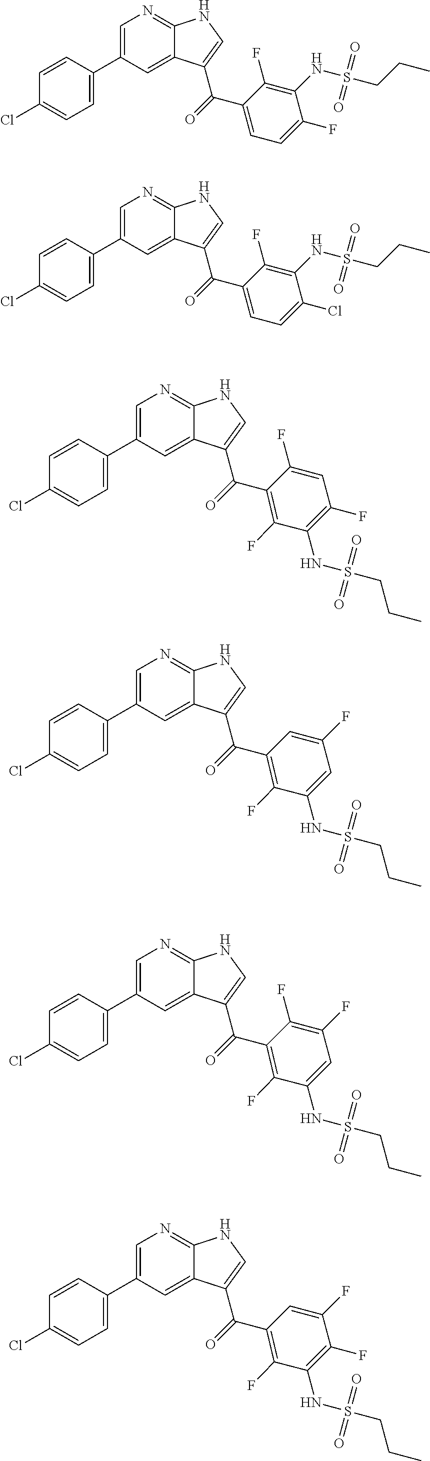 [SMILES: CCCS(=O)(=O)Nc1c(F)ccc(C(=O)c2c[nH]c3ncc(-c4ccc(Cl)cc4)cc23)c1F ; CCCS(=O)(=O)Nc1c(Cl)ccc(C(=O)c2c[nH]c3ncc(-c4ccc(Cl)cc4)cc23)c1F ; CCCS(=O)(=O)Nc1c(F)cc(F)c(C(=O)c2c[nH]c3ncc(-c4ccc(Cl)cc4)cc23)c1F ; CCCS(=O)(=O)Nc1cc(F)cc(C(=O)c2c[nH]c3ncc(-c4ccc(Cl)cc4)cc23)c1F ; CCCS(=O)(=O)Nc1cc(F)c(F)c(C(=O)c2c[nH]c3ncc(-c4ccc(Cl)cc4)cc23)c1F ; CCCS(=O)(=O)Nc1c(F)c(F)cc(C(=O)c2c[nH]c3ncc(-c4ccc(Cl)cc4)cc23)c1F]

N-(3-(5-(4-chlorophenyl)-1H-pyrrolo[2,3-b]pyridine-3-carbonyl)-5-fluorophenyl)propane-1-sulfonamide;
N-(2,4-difluoro-3-(5-(4-fluoro-2-methylphenyl)-1H-pyrrolo[2,3-b]pyridine-3-carbonyl)phenyl)butane-1-sulfonamide;
N-(3-(5-(4-(4-acetylpiperazin-1-yl)phenyl)-1H-pyrrolo[2,3-b]pyridine-3-carbonyl)-2,4-difluorophenyl)propane-1-sulfonamide;
N-(3-(5-(2-cyclopropylpyrimidin-5-yl)-1H-pyrrolo[2,3-b]pyridine-3-carbonyl)-2,6-difluorophenyl)-methanesulfonamide
N-(2,6-difluoro-3-(5-(2-(trifluoromethyl)pyrimidin-5-yl)-1H-pyrrolo[2,3-b]pyridine-3-carbonyl)phenyl)-3,3,3-trifluoro-propane-1-sulfonamide
N-(3-(5-(3-cyanophenyl)-1H-pyrrolo[2,3-b]pyridine-3-carbonyl)-2,6-difluorophenyl)propane-1-sulfonamide
N-(2,6-difluoro-3-(5-(pyridazin-4-yl)-1H-pyrrolo[2,3-b]pyridine-3-carbonyl)phenyl)propane-1-sulfonamide;
N-(2,6-difluoro-3-(5-(pyridazin-3-yl)-1H-pyrrolo[2,3-b]pyridine-3-carbonyl)phenyl)propane-1-sulfonamide;
N-(3-(5-(2-cyclopropyl-pyrimidin-5-yl)-1H-pyrrolo[2,3-b]pyridine-3-carbonyl)-2,6-difluorophenyl)-propane-1-sulfonamide;
N-(2,6-difluoro-3-(5-(2-methylpyrimidin-5-yl)-1H-pyrrolo-[2,3-b]pyridine-3-carbonyl)-phenyl)-3,3,3-trifluoropropane-1-sulfonamide;
N-(2,6-difluoro-3-(5-(6-(trifluoro-methyl)pyridin-3-yl)-1H-pyrrolo-[2,3-b]pyridine-3-carbonyl)-phenyl)-3,3,3-trifluoropropane-1-sulfonamide;
N-(3-(5-(4-chlorophenyl)-1H-pyrrolo[2,3-b]pyridine-3-carbonyl)-2,6-difluorophenyl)methane-sulfonamide;
N-(3-(5-(4-chlorophenyl)-1H-pyrrolo[2,3-b]pyridine-3-carbonyl)-2,6-difluorophenyl)-3,3,3-trifluoropropane-1-sulfonamide;
N-(2,6-difluoro-3-(5-(4-(S-methylsulfonimidoyl)phenyl)-1H-pyrrolo[2,3-b]pyridine-3-carbonyl)phenyl)propane-1-sulfonamide;
N-(3-(5-(2-chloro-4-hydroxyphenyl)-1H-pyrrolo[2,3-b]pyridine-3-carbonyl)-2,6-difluorophenyl)propane-1-sulfonamide;
N-(3-(5-(2-chloro-4-(2,3-dihydroxypropoxy)phenyl)-1H-pyrrolo[2,3-b]pyridine-3-carbonyl)-2,6-difluorophenyl)propane-1-sulfonamide;
N-(3-(5-(4-(2,3-dihydroxypropoxy)phenyl)-1H-pyrrolo[2,3-b]pyridine-3-carbonyl)-2,6-difluorophenyl)propane-1-sulfonamide;
N-(2,6-difluoro-3-(5-(4-(pentafluoro-16-sulfaneyl)phenyl)-1H-pyrrolo[2,3-b]pyridine-3-carbonyl)phenyl)propane-1-sulfonamide;
N-(2,6-difluoro-3-(5-(pyridin-3-yl)-1H-pyrrolo[2,3-b]pyridine-3-carbonyl)phenyl)-3,3,3-trifluoropropane-1-sulfonamide
N-(2,6-difluoro-3-(5-(6-fluoro-4-methylpyridin-3-yl)-1H-pyrrolo[2,3-b]pyridine-3-carbonyl)phenyl)-3,3,3-trifluoropropane-1-sulfonamide
N-(2,6-difluoro-3-(5-(6-methoxypyridin-3-yl)-1H-pyrrolo[2,3-b]pyridine-3-carbonyl)phenyl)-3,3,3-trifluoropropane-1-sulfonamide;
N-(3-(5-(6-cyclopropylpyridin-3-yl)-1H-pyrrolo[2,3-b]pyridine-3-carbonyl)-2,6-difluorophenyl)propane-1-sulfonamide;
N-(2,6-difluoro-3-(5-(6-(methylamino)pyridin-3-yl)-1H-pyrrolo[2,3-b]pyridine-3-carbonyl)phenyl)propane-1-sulfonamide;
N-(3-(5-(6-(dimethylamino)pyridin-3-yl)-1H-pyrrolo[2,3-b]pyridine-3-carbonyl)-2,6-difluorophenyl)propane-1-sulfonamide;
N-(3-(5-(6-(azetidin-1-yl)pyridin-3-yl)-1H-pyrrolo[2,3-b]pyridine-3-carbonyl)-2,6-difluorophenyl)propane-1-sulfonamide;
N-(2,6-difluoro-3-(5-(6-vinylpyridin-3-yl)-1H-pyrrolo[2,3-b]pyridine-3-carbonyl)phenyl)propane-1-sulfonamide;
N-(2,6-difluoro-3-(5-(6-(S-methylsulfonimidoyl)pyridin-3-yl)-1H-pyrrolo[2,3-b]pyridine-3-carbonyl)phenyl)propane-1-sulfonamide;
N-(3-(5-(2,6-dimethylpyridin-3-yl)-1H-pyrrolo[2,3-b]pyridine-3-carbonyl)-2,6-difluorophenyl)propane-1-sulfonamide:
N-(3-(5-(2-chloro-6-methylpyridin-3-yl)-1H-pyrrolo[2,3-b]pyridine-3-carbonyl)-2,6-difluorophenyl)propane-1-sulfonamide;
N-(3-(5-(6-cyclopropyl-4-methylpyridin-3-yl)-1H-pyrrolo[2,3-b]pyridine-3-carbonyl)-2,6-difluorophenyl)propane-1-sulfonamide;
N-(2,6-difluoro-3-(5-(2,4,6-trimethylpyridin-3-yl)-1H-pyrrolo[2,3-b]pyridine-3-carbonyl)phenyl)propane-1-sulfonamide;
N-(3-(5-(2-chloro-6-methylpyridin-3-yl)-1H-pyrrolo[2,3-b]pyridine-3-carbonyl)-2,6-difluorophenyl)propane-1-sulfonamide;
N-(3-(5-(6-cyclopropyl-2-methylpyridin-3-yl)-1H-pyrrolo[2,3-b]pyridine-3-carbonyl)-2,6-difluorophenyl)propane-1-sulfonamide;
N-(3-(5-(4,6-dimethylpyridin-3-yl)-1H-pyrrolo[2,3-b]pyridine-3-carbonyl)-2,6-difluorophenyl)propane-1-sulfonamide;
N-(2,6-difluoro-3-(5-(3-methylpyridin-4-yl)-1H-pyrrolo[2,3-b]pyridine-3-carbonyl)phenyl)-3,3,3-trifluoropropane-1-sulfonamide;
N-(3-(5-(3-chloropyridin-4-yl)-1H-pyrrolo[2,3-b]pyridine-3-carbonyl)-2,6-difluorophenyl)propane-1-sulfonamide;
N-(2,6-difluoro-3-(5-(3-fluoropyridin-4-yl)-1H-pyrrolo[2,3-b]pyridine-3-carbonyl)phenyl)propane-1-sulfonamide;
N-(3-(5-(2-cyclopropylpyrimidin-5-yl)-1H-pyrrolo[2,3-b]pyridine-3-carbonyl)-2,6-difluorophenyl)-3,3,3-trifluoropropane-1-sulfonamide;
N-(2,6-difluoro-3-(5-(2-(methylamino)pyrimidin-5-yl)-1H-pyrrolo[2,3-b]pyridine-3-carbonyl)phenyl)-3,3,3-trifluoropropane-1-sulfonamide;
N-(3-(5-(2-(dimethylamino)pyrimidin-5-yl)-1H-pyrrolo[2,3-b]pyridine-3-carbonyl)-2,6-difluorophenyl)-3,3,3-trifluoropropane-1-sulfonamide;
N-(2,6-difluoro-3-(5-(2-(pyrrolidin-1-yl)pyrimidin-5-yl)-1H-pyrrolo[2,3-b]pyridine-3-carbonyl)phenyl)-3,3,3-trifluoropropane-1-sulfonamide;
N-(3-(5-(2-(azetidin-1-yl)pyrimidin-5-yl)-1H-pyrrolo[2,3-b]pyridine-3-carbonyl)-2,6-difluorophenyl)-3,3,3-trifluoropropane-1-sulfonamide;
N-(2,6-difluoro-3-(5-(2-(piperidin-1-yl)pyrimidin-5-yl)-1H-pyrrolo[2,3-b]pyridine-3-carbonyl)phenyl)-3,3,3-trifluoropropane-1-sulfonamide;
N-(3-(5-(2-(cyclopropylamino)pyrimidin-5-yl)-1H-pyrrolo[2,3-b]pyridine-3-carbonyl)-2,6-difluorophenyl)-3,3,3-trifluoropropane-1-sulfonamide;
N-(3-(5-(2-cyclopropylpyrimidin-5-yl)-1H-pyrrolo[2,3-b]pyridine-3-carbonyl)-2,6-difluorophenyl)ethanesulfonamide;
N-(2,6-difluoro-3-(5-(2-(trifluoromethyl)pyrimidin-5-yl)-1H-pyrrolo[2,3-b]pyridine-3-carbonyl)phenyl)propane-1-sulfonamide;
N-(2,6-difluoro-3-(5-(2-(piperazin-1-yl)pyrimidin-5-yl)-1H-pyrrolo[2,3-b]pyridine-3-carbonyl)phenyl)propane-1-sulfonamide;
N-(3-(5-(4-chloro-2-methylpyrimidin-5-yl)-1H-pyrrolo[2,3-b]pyridine-3-carbonyl)-2,6-difluorophenyl)propane-1-sulfonamide
N-(2,6-difluoro-3-(5-(2-((2-methoxyethyl)amino)pyrimidin-5-yl)-1H-pyrrolo[2,3-b]pyridine-3-carbonyl)phenyl)propane-1-sulfonamide;
N-(2,6-difluoro-3-(5-(2-morpholinopyrimidin-5-yl)-1H-pyrrolo[2,3-b]pyridine-3-carbonyl)phenyl)propane-1-sulfonamide;
N-(3-(5-(2-cyanopyrimidin-5-yl)-1H-pyrrolo[2,3-b]pyridine-3-carbonyl)-2,6-difluorophenyl)propane-1-sulfonamide;
5-(3-(2,4-difluoro-3-(propylsulfonamido)benzoyl)-1H-pyrrolo[2,3-b]pyridin-5-yl)pyrimidine-2-carboxamide;
N-(2,6-difluoro-3-(5-(2-(pyrrolidin-1-yl)pyrimidin-5-yl)-1H-pyrrolo[2,3-b]pyridine-3-carbonyl)phenyl)propane-1-sulfonamide;
N-(2,6-difluoro-3-(5-(2-((3-methoxypropyl)amino)pyrimidin-5-yl)-1H-pyrrolo[2,3-b]pyridine-3-carbonyl)phenyl)propane-1-sulfonamide;
N-(2,6-difluoro-3-(5-(2-isopropylpyrimidin-5-yl)-1H-pyrrolo[2,3-b]pyridine-3-carbonyl)phenyl)propane-1-sulfonamide;
N-(2,6-difluoro-3-(5-(2-(4-methylpiperazin-1-yl)pyrimidin-5-yl)-1H-pyrrolo[2,3-b]pyridine-3-carbonyl)phenyl)propane-1-sulfonamide;
N-(2,6-difluoro-3-(5-(2-(methylthio)pyrimidin-5-yl)-1H-pyrrolo[2,3-b]pyridine-3-carbonyl)phenyl)propane-1-sulfonamide;
N-(3-(5-(2-(tert-butyl)pyrimidin-5-yl)-1H-pyrrolo[2,3-b]pyridine-3-carbonyl)-2,6-difluorophenyl)propane-1-sulfonamide;
N-(2,6-difluoro-3-(5-(2-(isopropylthio)pyrimidin-5-yl)-1H-pyrrolo[2,3-b]pyridine-3-carbonyl)phenyl)propane-1-sulfonamide;
N-(2,6-difluoro-3-(5-(2-(4-(2-hydroxyethyl)piperazin-1-yl)pyrimidin-5-yl)-1H-pyrrolo[2,3-b]pyridine-3-carbonyl)phenyl)propane-1-sulfonamide;
N-(3-(5-(2-(azetidin-1-yl)pyrimidin-5-yl)-1H-pyrrolo[2,3-b]pyridine-3-carbonyl)-2,6-difluorophenyl)propane-1-sulfonamide;
N-(3-(5-(2-(3-(dimethylamino)propoxy)pyrimidin-5-yl)-1H-pyrrolo[2,3-b]pyridine-3-carbonyl)-2,6-difluorophenyl)propane-1-sulfonamide;
N-(2,6-difluoro-3-(5-(2-((2-hydroxyethyl)amino)pyrimidin-5-yl)-1H-pyrrolo[2,3-b]pyridine-3-carbonyl)phenyl)propane-1-sulfonamide;
N-(2,6-difluoro-3-(5-(2-((2-hydroxyethyl)(methyl)amino)pyrimidin-5-yl)-1H-pyrrolo[2,3-b]pyridine-3-carbonyl)phenyl)propane-1-sulfonamide;
(S)—N-(2,6-difluoro-3-(5-(2-(3-hydroxypyrrolidin-1-yl)pyrimidin-5-yl)-1H-pyrrolo[2,3-b]pyridine-3-carbonyl)phenyl)propane-1-sulfonamide;
(R)—N-(2,6-difluoro-3-(5-(2-(3-hydroxypyrrolidin-1-yl)pyrimidin-5-yl)-1H-pyrrolo[2,3-b]pyridine-3-carbonyl)phenyl)propane-1-sulfonamide;
N-(2,6-difluoro-3-(5-(2-(3-hydroxyazetidin-1-yl)pyrimidin-5-yl)-1H-pyrrolo[2,3-b]pyridine-3-carbonyl)phenyl)propane-1-sulfonamide;
N-(3-(5-(2-ethylpyrimidin-5-yl)-1H-pyrrolo[2,3-b]pyridine-3-carbonyl)-2,6-difluorophenyl)propane-1-sulfonamide;
N-(2,6-difluoro-3-(5-(2-(fluoromethyl)pyrimidin-5-yl)-1H-pyrrolo[2,3-b]pyridine-3-carbonyl)phenyl)propane-1-sulfonamide;
N-(3-(5-(2-ethynylpyrimidin-5-yl)-1H-pyrrolo[2,3-b]pyridine-3-carbonyl)-2,6-difluorophenyl)propane-1-sulfonamide;
N-(2,6-difluoro-3-(5-(4-methyl-2-(methylthio)pyrimidin-5-yl)-1H-pyrrolo[2,3-b]pyridine-3-carbonyl)phenyl)propane-1-sulfonamide;
N-(3-(5-(2-(1H-benzo[d]imidazol-6-yl)pyrimidin-5-yl)-1H-pyrrolo[2,3-b]pyridine-3-carbonyl)-2,6-difluorophenyl)propane-1-sulfonamide;
N-(3-(5-(5-chloropyrazin-2-yl)-1H-pyrrolo[2,3-b]pyridine-3-carbonyl)-2,6-difluorophenyl)propane-1-sulfonamide;
(3-amino-2,4-difluorophenyl)(5-(4-chlorophenyl)-1H-pyrrolo[2,3-b]pyridin-3-yl)methanone;
N-(3-(5-(2-chlorophenyl)-1H-pyrrolo[2,3-b]pyridine-3-carbonyl)-2,6-difluorophenyl)propane-1-sulfonamide;
N-(2,6-difluoro-3-(5-(naphthalen-1-yl)-1H-pyrrolo[2,3-b]pyridine-3-carbonyl)phenyl)methanesulfonamide;
N-(2,6-difluoro-3-(5-(o-tolyl)-1H-pyrrolo[2,3-b]pyridine-3-carbonyl)phenyl)propane-1-sulfonamide;
N-(2,6-difluoro-3-(5-(4-fluorophenyl)-1H-pyrrolo[2,3-b]pyridine-3-carbonyl)phenyl)propane-1-sulfonamide;
N-(2,6-difluoro-3-(5-(4-fluoro-2-methylphenyl)-1H-pyrrolo[2,3-b]pyridine-3-carbonyl)phenyl)propane-1-sulfonamide;
N-(3-(5-(2-chloro-4-methoxyphenyl)-1H-pyrrolo[2,3-b]pyridine-3-carbonyl)-2,6-difluorophenyl)propane-1-sulfonamide;
N-(3-(5-(4-(1H-tetrazol-5-yl)phenyl)-1H-pyrrolo[2,3-b]pyridine-3-carbonyl)-2,6-difluorophenyl)propane-1-sulfonamide;
4-(3-(2,4-difluoro-3-(propylsulfonamido)benzoyl)-1H-pyrrolo[2,3-b]pyridin-5-yl)benzenesulfonamide;
N-(3-(5-(2-chloro-4-fluorophenyl)-1H-pyrrolo[2,3-b]pyridine-3-carbonyl)-2,6-difluorophenyl)propane-1-sulfonamide:
4-(3-(2,4-difluoro-3-(propylsulfonamido)benzoyl)-1H-pyrrolo[2,3-b]pyridin-5-yl)benzoic acid;
3-(3-(2,4-difluoro-3-(propylsulfonamido)benzoyl)-1H-pyrrolo[2,3-b]pyridin-5-yl)benzoic acid;
2-(3-(2,4-difluoro-3-(propylsulfonamido)benzoyl)-1H-pyrrolo[2,3-b]pyridin-5-yl)benzoic acid;
N-(3-(5-(4-chloro-2-methylphenyl)-1H-pyrrolo[2,3-b]pyridine-3-carbonyl)-2,6-difluorophenyl)propane-1-sulfonamide;
N-(3-(5-(2-chloro-4-methoxyphenyl)-1H-pyrrolo[2,3-b]pyridine-3-carbonyl)-2,6-difluorophenyl)butane-1-sulfonamide;
N-(2,6-difluoro-3-(5-(4-fluoro-2-methylphenyl)-1H-pyrrolo[2,3-b]pyridine-3-carbonyl)phenyl)butane-1-sulfonamide;
N-(2,6-difluoro-3-(5-(pyridin-3-yl)-1H-pyrrolo[2,3-b]pyridine-3-carbonyl)phenyl)methanesulfonamide;
N-(2,6-difluoro-3-(5-(6-methylpyridin-3-yl)-1H-pyrrolo[2,3-b]pyridine-3-carbonyl)phenyl)propane-1-sulfonamide;
N-(2,6-difluoro-3-(5-(6-(trifluoromethyl)pyridin-3-yl)-1H-pyrrolo[2,3-b]pyridine-3-carbonyl)phenyl)propane-1-sulfonamide;
N-(2,6-difluoro-3-(5-(pyridin-4-yl)-1H-pyrrolo[2,3-b]pyridine-3-carbonyl)phenyl)ethane sulfonamide;
N-(2,6-difluoro-3-(5-(pyridin-3-yl)-1H-pyrrolo[2,3-b]pyridine-3-carbonyl)phenyl)propane-1-sulfonamide;
N-(2,6-difluoro-3-(5-(pyridin-4-yl)-1H-pyrrolo[2,3-b]pyridine-3-carbonyl)phenyl)propane-1-sulfonamide;
N-(2,6-difluoro-3-(5-(pyridin-4-yl)-1H-pyrrolo[2,3-b]pyridine-3-carbonyl)phenyl)methanesulfonamide;
N-(2,6-difluoro-3-(5-(3-methylpyridin-4-yl)-1H-pyrrolo[2,3-b]pyridine-3-carbonyl)phenyl)propane-1-sulfonamide;
N-(3-(5-(2,4-dimethoxypyrimidin-5-yl)-1H-pyrrolo[2,3-b]pyridine-3-carbonyl)-2,6-difluorophenyl)propane-1-sulfonamide;
N-(3-(5-(2-chloropyrimidin-5-yl)-1H-pyrrolo[2,3-b]pyridine-3-carbonyl)-2,6-difluorophenyl)propane-1-sulfonamide;
N-(2,6-difluoro-3-(5-(2-methoxypyrimidin-5-yl)-1H-pyrrolo[2,3-b]pyridine-3-carbonyl)phenyl)propane-1-sulfonamide;
N-(3-(5-cyclobutyl-1H-pyrrolo[2,3-b]pyridine-3-carbonyl)-2,6-difluorophenyl)propane-1-sulfonamide;
N-(3-(5-(2-chloro-4-methoxyphenyl)-1H-pyrazolo[3,4-b]pyridine-3-carbonyl)-2-fluorophenyl)-methane-sulfonamide
N-(2-fluoro-3-(5-(4-fluoro-2-methylphenyl)-1H-pyrazolo[3,4-b]pyridine-3-carbonyl)-phenyl)methanesulfonamide;
N-(3-(5-(2,3-dihydro-benzo-[b][1,4]dioxin-6-yl)-1H-pyrazolo[3,4-b]pyridine-3-carbonyl)-2-fluoro-phenyl)-methanesulfonamide;
N-(3-(5-(2-chloro-4-methoxy-phenyl)-1H-pyrazolo[3,4-b]pyridine-3-carbonyl)-2-fluorophenyl)-butane-1-sulfonamide;
N-(2-fluoro-3-(5-(4-fluoro-2-methylphenyl)-1H-pyrazo-lo[3,4-b]pyridine-3-carbonyl)-phenyl)butane-1-sulfonamide;
N-(3-(5-(2,3-dihydro-benzo-[b][1,4]dioxin-6-yl)-1H-pyrazolo[3,4-b]-pyridine-3-carbonyl)-2-fluorophenyl)-butane-1-sulfonamide;
N-[2,6-difluoro-3-[5-[4-(1H-tetrazol-5-yl)phenyl]-1H-pyrazolo[3,4-b]pyridine-3-carbonyl]phenyl]methanesulfonamide;
4-[3-[2,4-difluoro-3-(methanesulfonamido)benzoyl]-1H-pyrazolo[3,4-b]pyridin-5-yl]benzamide;
4-[3-[2,4-difluoro-3-(methanesulfonamido)benzoyl]-1H-pyrazolo[3,4-b]pyridin-5-yl]benzenesulfonamide;
4-[3-[2,4-difluoro-3-(methanesulfonamido)benzoyl]-1H-pyrazolo[3,4-b]pyridin-5-yl]benzoic acid;
ethyl 4-[3-[2,4-difluoro-3-(methanesulfonamido)benzoyl]-1H-pyrazolo[3,4-b]pyridin-5-yl]benzoate;
[(2S)-2-amino-3-methoxy-3-oxopropyl]4-[3-[2,4-difluoro-3-(methanesulfonamido)benzoyl]-1H-pyrazolo[3,4-b]pyridin-5-yl]benzoate;
(2S)-2-amino-3-[4-[3-[2,4-difluoro-3-(methanesulfonamido)-benzoyl]-1H-pyrazolo[3,4-b]pyridin-5-yl]benzoyl]oxypropanoic acid;
5-[3-[2,4-difluoro-3-(methanesulfonamido)benzoyl]-1H-pyrazolo[3,4-b]pyridin-5-yl]pyridine-2-carboxylic acid;
N-[2,6-difluoro-3-(5-pyridin-4-yl-1H-pyrazolo[3,4-b]pyridine-3-carbonyl)phenyl]methanesulfonamide;
N-[2,6-difluoro-3-[5-[4-(methylsulfonimidoyl)phenyl]-1H-pyrazolo[3,4-b]pyridine-3-carbonyl]phenyl]methanesulfonamide;
N-[3-[5-(4-chlorophenyl)-1H-pyrazolo[3,4-b]pyridine-3-carbonyl]-2,6-difluorophenyl]propane-1-sulfonamide;
4-(3-(2,4-difluoro-3-(propylsulfon-amido)benzoyl)-1H-pyrazolo[3,4-b]pyridin-5-yl)benzoic acid;
N-(2,6-difluoro-3-(5-(pyridin-4-yl)-1H-pyrazolo[3,4-b]pyridine-3-carbonyl)phenyl)propane-1-sulfonamide;
N-(3-(5-(2-chlorophenyl)-1H-pyrazolo[3,4-b]pyridine-3-carbonyl)-2,6-difluorophenyl)propane-1-sulfonamide;
N-(2,6-difluoro-3-(5-(4-isopropyl-phenyl)-1H-pyrazolo[3,4-b]pyridine-3-carbonyl)phenyl)propane-1-sulfonamide;
N-(2,6-difluoro-3-(5-(4-fluoro-2-methylphenyl)-1H-pyrazolo[3,4-b]pyridine-3-carbonyl)phenyl) propane-1-sulfonamide;
4-(3-(2,4-difluoro-3-(propylsulfon-amido)benzoyl)-1H-pyrazolo[3,4-b]pyridin-5-yl)benzenesulfonamide;
N-[2,6-difluoro-3-(5-pyridin-4-yl-1H-pyrazolo[3,4-b]pyridine-3-carbonyl)phenyl]ethanesulfonamide;
4-(3-(2,4-difluoro-3-(propylsulfonamido)benzoyl)-1H-pyrazolo[3,4-b]pyridin-5-yl)-3-methylbenzenesulfonamide;
N-(2,6-difluoro-3-(5-(2-(trifluoromethyl)pyrimidin-5-yl)-1H-pyrazolo[3,4-b]pyridine-3-carbonyl)phenyl)propane-1-sulfonamide;
N-(3-(5-(4-chloro-2-methylphenyl)-1H-pyrazolo[3,4-b]pyridine-3-carbonyl)-2,6-difluorophenyl)propane-1-sulfonamide;
N-(3-(5-(4-(1H-tetrazol-5-yl)phenyl)-1H-pyrazolo[3,4-b]pyridine-3-carbonyl)-2,6-difluorophenyl)ethanesulfonamide;
N-(3-(5-(4-(1H-tetrazol-5-yl)phenyl)-1H-pyrazolo[3,4-b]pyridine-3-carbonyl)-2,6-difluorophenyl)propane-1-sulfonamide;
N-(2,6-difluoro-3-(5-(4-hydroxyphenyl)-1H-pyrazolo[3,4-b]pyridine-3-carbonyl)phenyl)propane-1-sulfonamide;

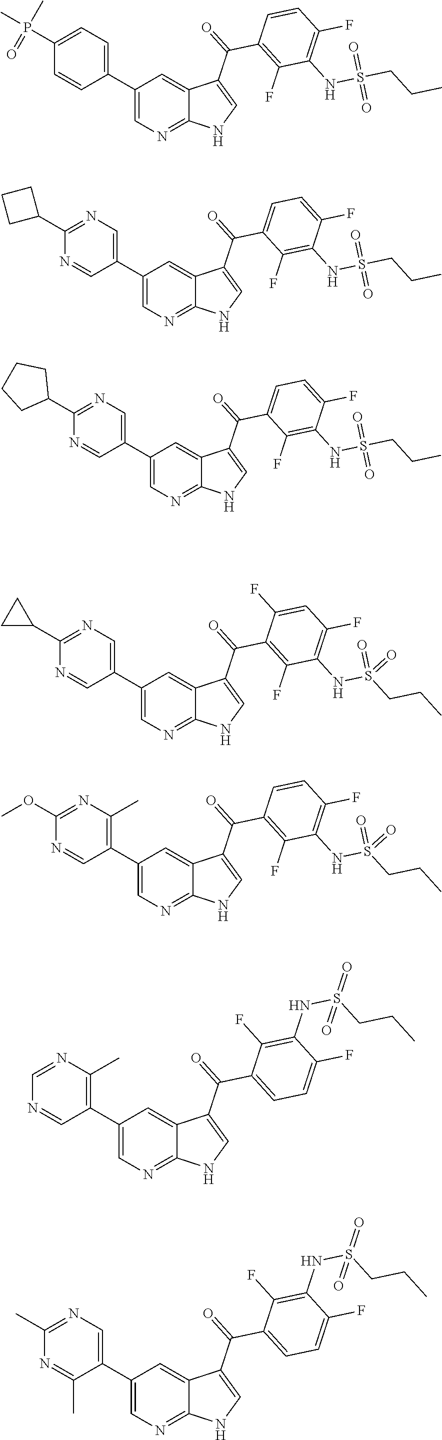

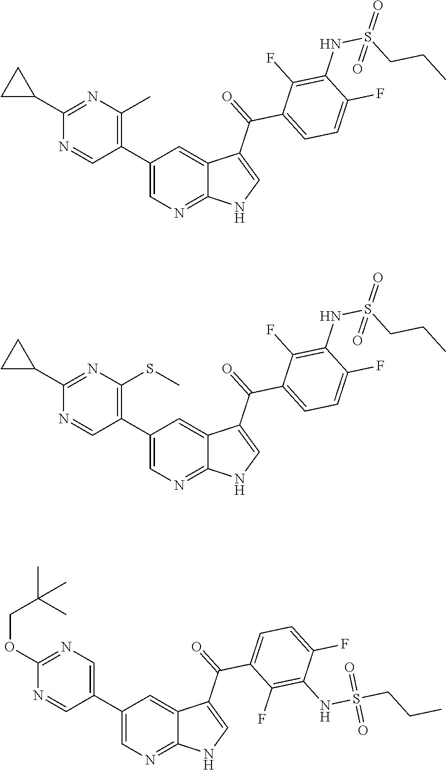 [SMILES: CCCS(=O)(=O)Nc1c(F)ccc(C(=O)c2c[nH]c3ncc(-c4cnc(C5CC5)nc4C)cc23)c1F ; CCCS(=O)(=O)Nc1c(F)ccc(C(=O)c2c[nH]c3ncc(-c4cnc(C5CC5)nc4SC)cc23)c1F ; CCCS(=O)(=O)Nc1c(F)ccc(C(=O)c2c[nH]c3ncc(-c4cnc(OCC(C)(C)C)nc4)cc23)c1F]

N-(3-(5-(4-chlorophenyl)-1H-pyrazolo[3,4-b]pyridine-3-carbonyl)-2,6-difluorophenyl)methanesulfonamide;
N-(3-(5-(4-chlorophenyl)-1H-pyrazolo[3,4-b]pyridine-3-carbonyl)-2,6-difluorophenyl)ethanesulfonamide;
ethyl 4-(3-(2,4-difluoro-3-(methylsulfonamido)benzoyl)-1H-pyrazolo[3,4-b]pyridin-5-yl)benzoate;
methyl 5-(3-(2,4-difluoro-3-(methylsulfonamido)benzoyl)-1H-pyrazolo[3,4-b]pyridin-5-yl)picolinate;
N-(2,6-difluoro-3-(5-(pyrimidin-5-yl)-1H-pyrazolo[3,4-b]pyridine-3-carbonyl)phenyl)propane-1-sulfonamide;
N-(2,6-difluoro-3-(5-(2-methylpyrimidin-5-yl)-1H-pyrazolo[3,4-b]pyridine-3-carbonyl)phenyl)propane-1-sulfonamide;
5-(3-(2,4-difluoro-3-(propylsulfonamido)benzoyl)-1H-pyrazolo[3,4-b]pyridin-5-yl)pyrimidine-2-carboxylic acid;
N-(3-(5-(2-cyanopyrimidin-5-yl)-1H-pyrazolo[3,4-b]pyridine-3-carbonyl)-2,6-difluorophenyl)propane-1-sulfonamide;
N-(3-(5-(2-(1H-tetrazol-5-yl)pyrimidin-5-yl)-1H-pyrazolo[3,4-b]pyridine-3-carbonyl)-2,6-difluorophenyl)propane-1-sulfonamide;
N-(3-(5-(2-cyclopropylpyrimidin-5-yl)-1H-pyrazolo[3,4-b]pyridine-3-carbonyl)-2,6-difluorophenyl)propane-1-sulfonamide;
N-(3-(5-(2-chloropyrimidin-5-yl)-1H-pyrazolo[3,4-b]pyridine-3-carbonyl)-2,6-difluorophenyl)propane-1-sulfonamide;
N-(3-(5-(4-chlorophenyl)-1H-pyrazolo[3,4-b]pyridine-3-carbonyl)-2,4,6-trifluorophenyl)propane-1-sulfonamide;
N-(2,6-difluoro-3-(5-(2-methoxypyrimidin-5-yl)-1H-pyrazolo[3,4-b]pyridine-3-carbonyl)phenyl)propane-1-sulfonamide;
N-(2,6-difluoro-3-(5-(2-hydroxypyrimidin-5-yl)-1H-pyrazolo[3,4-b]pyridine-3-carbonyl)phenyl)propane-1-sulfonamide;
N-(3-(5-(3-chloropyridin-4-yl)-1H-pyrazolo[3,4-b]pyridine-3-carbonyl)-2,6-difluorophenyl)propane-1-sulfonamide;
N-(2,6-difluoro-3-(5-(3-methylpyridin-4-yl)-1H-pyrazolo[3,4-b]pyridine-3-carbonyl)phenyl)propane-1-sulfonamide;
N-(3-(5-(4-(tert-butyl)phenyl)-1H-pyrazolo[3,4-b]pyridine-3-carbonyl)-2,6-difluorophenyl)propane-1-sulfonamide;
N-(3-(5-(2-chloro-4-methoxyphenyl)-1H-pyrazolo[3,4-b]pyridine-3-carbonyl)-2,6-difluorophenyl)propane-1-sulfonamide;
N-(3-(5-(2-(tert-butyl)pyrimidin-5-yl)-1H-pyrazolo[3,4-b]pyridine-3-carbonyl)-2,6-difluorophenyl)propane-1-sulfonamide;
N-(2,6-difluoro-3-(5-(2-(methylthio)pyrimidin-5-yl)-1H-pyrazolo[3,4-b]pyridine-3-carbonyl)phenyl)propane-1-sulfonamide;
N-(2,6-difluoro-3-(5-(2-isopropylpyrimidin-5-yl)-1H-pyrazolo[3,4-b]pyridine-3-carbonyl)phenyl)propane-1-sulfonamide;
N-(2,6-difluoro-3-(5-(4-fluoro-2-methylphenyl)-1H-pyrazolo[3,4-b]pyridine-3-carbonyl)phenyl)propane-1-sulfonamide;
N-(2,6-difluoro-3-(5-(4-methoxy-2-methylpyrimidin-5-yl)-1H-pyrazolo[3,4-b]pyridine-3-carbonyl)phenyl)propane-1-sulfonamide;
N-(2,6-difluoro-3-(5-(4-hydroxy-2-methylpyrimidin-5-yl)-1H-pyrazolo[3,4-b]pyridine-3-carbonyl)phenyl)propane-1-sulfonamide;
N-(2,6-difluoro-3-(5-(pyridin-3-yl)-1H-pyrazolo[3,4-b]pyridine-3-carbonyl)phenyl)propane-1-sulfonamide;
5-(3-(2,4-difluoro-3-(propylsulfonamido)benzoyl)-1H-pyrazolo[3,4-b]pyridin-5-yl)picolinic acid
N-[2,6-difluoro-3-[5-(4-methylpyrimidin-5-yl)-1H-pyrazolo[3,4-b]pyridine-3-carbonyl]phenyl]propane-1-sulfonamide;
N-[3-[5-(2,4-dimethylpyrimidin-5-yl)-1H-pyrazolo[3,4-b]pyridine-3-carbonyl]-2,6-difluoro-phenyl]propane-1-sulfonamide;
N-(3-(5-(2-cyclopropyl-4-methylpyrimidin-5-yl)-1H-pyrazolo[3,4-b]pyridine-3 carbonyl)-2,6-difluorophenyl)propane-1-sulfonamide;
N-(3-(5-(2-cyclopropyl-4-(methylthio)pyrimidin-5-yl)-1H-pyrazolo[3,4-b]pyridine-3 carbonyl)-2,6-difluorophenyl)propane-1-sulfonamide;
N-[2,6-difluoro-3-[5-(4-methyl-2-methylsulfanyl-pyrimidin-5-yl)-1H-pyrazolo[3,4-b]pyridine-3-carbonyl]phenyl]propane-1-sulfonamide;
N-[3-[5-(2,4-dimethoxypyrimidin-5-yl)-1H-pyrazolo[3,4-b]pyridine-3-carbonyl]-2,6-difluoro-phenyl]propane-1-sulfonamide;
N-(3-(5-(2-cyclopropylpyrimidin-5-yl)-1H-pyrazolo[3,4-b]pyridine-3-carbonyl)-2,4,6-trifluorophenyl)propane-1-sulfonamide;
N-[3-[5-(3-ethyl-4-pyridyl)-1H-pyrazolo[3,4-b]pyridine-3-carbonyl]-2,6-difluoro-phenyl]propane-1-sulfonamide;
N-[3-[5-(3-cyano-4-pyridyl)-1H-pyrazolo[3,4-b]pyridine-3-carbonyl]-2,6-difluoro-phenyl]propane-1-sulfonamide.

The following examples illustrate the invention without limiting it.

### Experimental Part

### A. Materials

### a) Chemicals

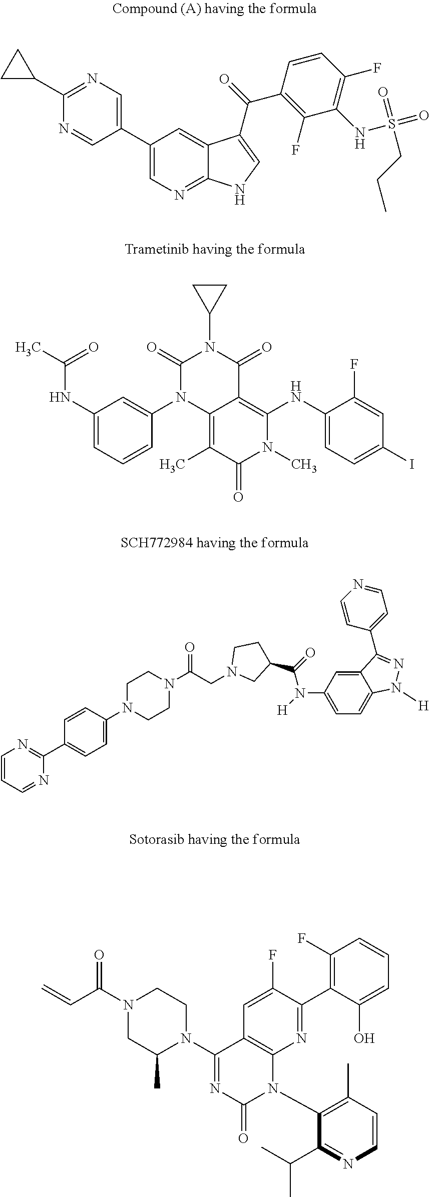

### b) Cell Lines

KRAS mutant lung and colorectal cancer cells:
Lung: H358 (G12C) (ATCC No: CRL-5807)
   H2122 (G12C) (ATCC No: CRL-5985)
Colon:DLD1 (G13D) (ATCC No: CCL-221)
   HCT 116 (G13D) (ATCC No: CCL-247)

### c) Cell Culture

All cell lines were cultured in RPMI 1640 medium (Gibco) supplemented with 10% fetal bovine serum, 1% glutamine and penicillin-streptomycin (Gibco) at 37° C. and 5% CO₂. Mycoplasma contamination was excluded via a PCR-based method.

### d) Analytical Methods

### Assay Method A—Colony Formation Matrix

Cell lines were cultured and seeded into 24-well plates. After 24 h incubation, drugs were added using the HP D300 Digital Dispenser. Medium and drugs were refreshed twice a week for 10-14 days. After this, cells were fixed with 4% formaldehyde in PBS and stained with 0.1% crystal violet in water. The quantification of the colony formation matrixes (crystal violet staining per well) was assessed using ImageJ software.

### Assay Method B—Western Blotting

Cells were washed with cold PBS and lysed with RIPA buffer supplemented with complete protease inhibitor (Roche) and phosphatase inhibitor cocktails II and Ill (Sigma). The protein concentration of the samples was normalized after performing a bicinchoninic acid assay (Pierce BCA, Thermo Scientific), according to the manufacturer's instructions. All lysates were freshly prepared and processed with Novex NuPAGE Gel Electrophoresis Systems (Thermo Fisher Scientific) followed by transferring to a polyvinylidene fluoride (PVDF) membrane. Then, membranes were placed in 5% bovine serum albumin for 1 h. Subsequently, membranes were probed with primary antibody and HRP-conjugated secondary antibody. Finally, a chemiluminescence substrate (ECL, Bio-Rad) was added to the membranes and the western blot was resolved using a ChemiDoc (Bio-Rad).

### Assay Method C—Proliferation and Caspase 3/7 Assay

For IncuCyte proliferation assays and Caspase 3/7 assays, cells were seeded in 96-well plates. After 24 h incubation, drugs and the IncuCyte® Caspase-3/7 green apoptosis assays reagent (Essen Bioscience 4440) were added using the HP D300 Digital Dispenser and the plates were placed in the IncuCyte®. Culture media and drugs were refreshed twice a week for 10 days. Cells were imaged every 4 hours in the IncuCyte ZOOM (Essen Bioscience). Phase-contrast images were collected and analyzed to detect cell proliferation based on cell confluence. Apoptosis was measured based on green fluorescent staining of apoptotic cells using the IncuCyte® Caspase-3/7 green apoptosis assay reagent (Essen Bioscience 4440).

### B. Examples

### Example 1: Compound (A) Synergizes with MEK Inhibition in KRAS Mutant Lung and Colorectal Cancer Cells

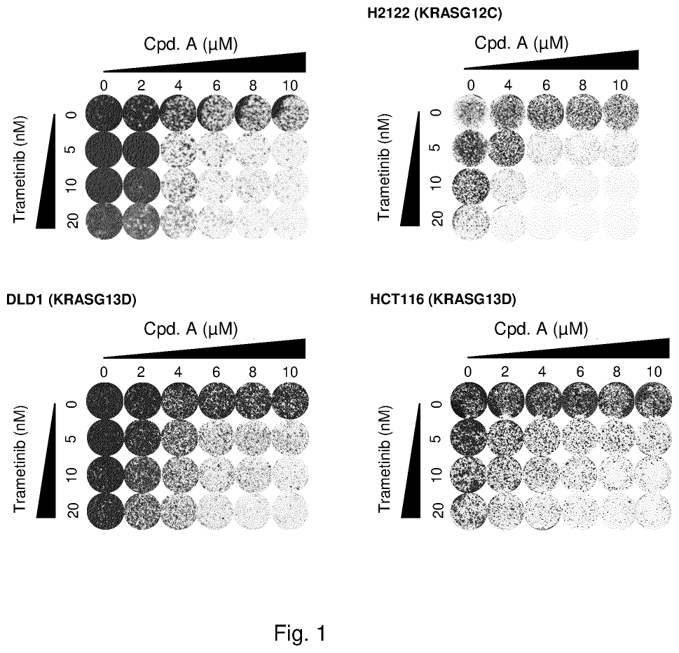

According to Assay Method A cells (H358, H2122, DLD1, HCT 116) were treated with compound (A), Trametinib or their combination at the indicated concentrations (cf. FIG. 1). Drugs were refreshed twice a week. The cells were fixed and stained after 10 days. The results are shown in FIG. 1.

### Example 2: Compound (A) Synergizes with ERK Inhibition in KRAS Mutant Lung and Colorectal Cancer Cells

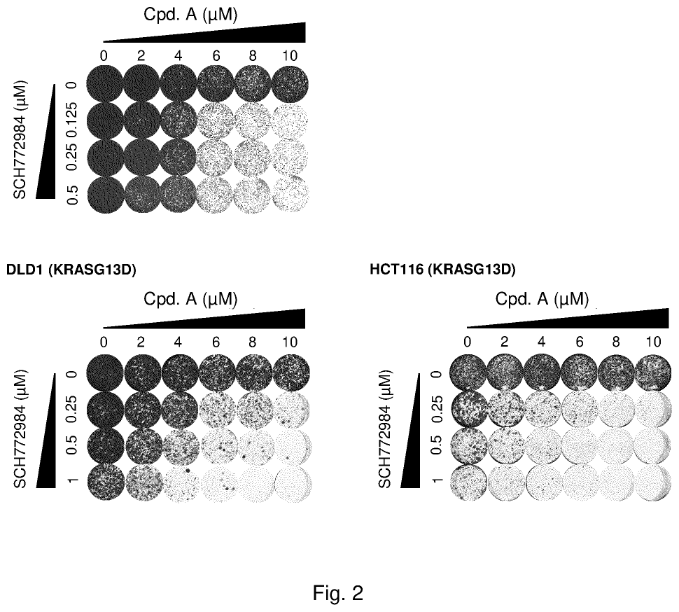

According to Assay Method A cells (H358, DLD1, HCT 116) were treated with compound (A), SCH772984 or their combination at the indicated concentrations (cf. FIG. 2). Drugs were refreshed twice a week. The cells were fixed and stained after 10 days. The results are shown in FIG. 2.

### Example 3: Compound (A) Synergizes with AMG510 in KRASG12C Mutant Lung Cancer Cells

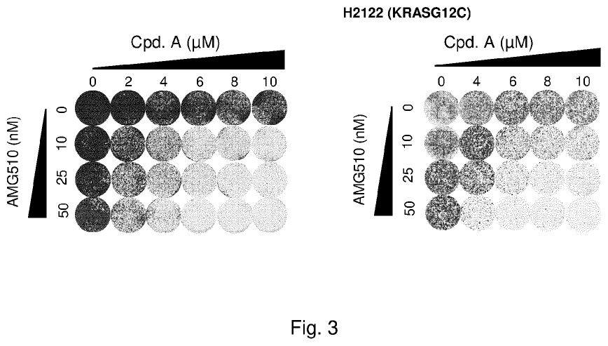

According to Assay Method A cells (H358, H2122) were treated with compound (A), AMG510 or their combination at the indicated concentrations (cf. FIG. 3). Drugs were refreshed twice a week. The cells were fixed and stained after 10 days. The results are shown in FIG. 3.

### Example 4: Compound (A) Synergizes with SHP2 Inhibition in KRASG12C Mutant Lung Cancer Cells

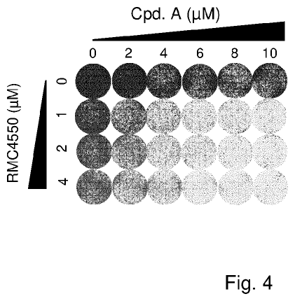

According to Assay Method A H358 cells were treated with compound (A), RMC4550 or their combination at the indicated concentrations (cf. FIG. 4). Drugs were refreshed twice a week. The cells were fixed and stained after 10 days. The results are shown in FIG. 4.

### Example 5: Compound (A) in Combination with MEK, SHP2 and KRAS^{G12C} Inhibition Abrogates Cell Proliferations and Induces Apoptosis in Lung Cancer Cells

According to assay method C cells were treated with compound (A) as component a) and RMC4550, AMG510, or Trametinib as component b) or combinations of component a) and either one of component b) at the indicated concentrations. Additionally, during the first treatment, the IncuCyte® Caspase-3/7 green apoptosis assay reagent was added to measure apoptosis for the first 72 hours. Drugs were refreshed twice a week. Confluence (left) and Caspase 3/7 activity was measured by the IncuCyte®. Standard error of the mean (SEM) from 3 independent replicates is plotted.

Further, Trametinib did not cause toxicity in the KRAS mutant cancer cells. Additionally, compound (A) alone also did not show sensitivity in these cells. The combination of compound (A) and Trametinib does show increased toxicity compared to the single drugs in these colony formation matrixes indicating synergy between MAP2K4 and MEK inhibition. These plots show a strong and wide range of synergy in the different mutants. Similar experiments were performed for the combination of the ERK inhibitor SCH772984 and compound (A).

As shown in example 3, the combination with compound (A) showed a strong synergy in these cancer cells (FIG. 3). Compared to the single agent effect of AMG510, addition of compound (A) strongly increased the toxicity across these KRAS G12C mutants.

Many KRAS-driven cancers rely on upstream signaling from proteins, such as SHP2. As shown in example 4, inhibition of SHP2, which is upstream of KRAS, combined with MAP2K4 inhibition strongly synergizes in the KRAS G12C mutant cancer cell line H358 (FIG. 4).

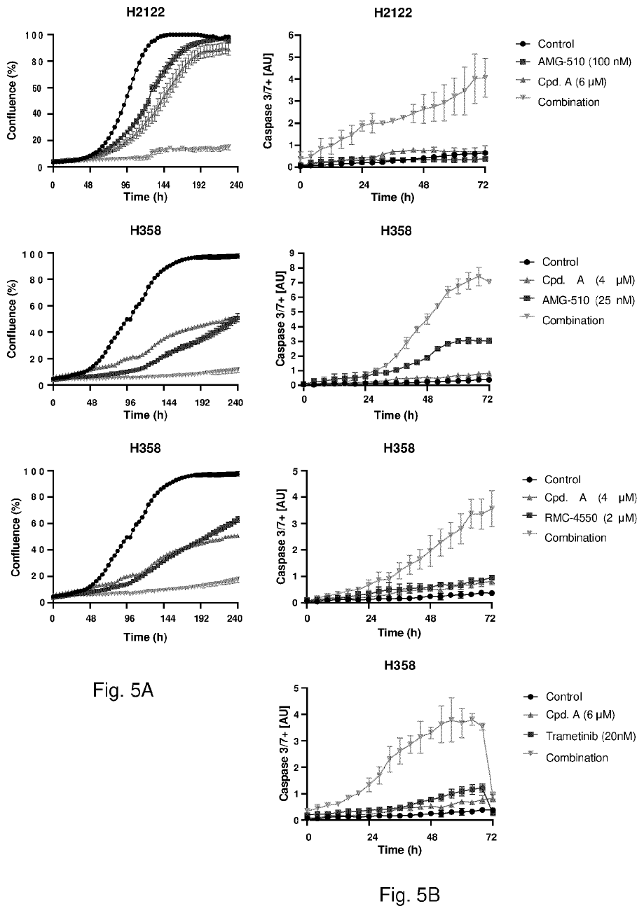

The synergistic effect achieved by a combination of compound (A) with MEK, SHP2 and KRASG12C inhibitors was confirmed by the results given in example 5 and FIGS. 5A and 5B.

Consistently, the obtained results demonstrate that compound (A) strongly synergizes with MEK, ERK, KRAS and SHP2 inhibition.

1. A pharmaceutical composition comprising a component a) and a component b), wherein
component a) comprises at least one compound having formula (I) 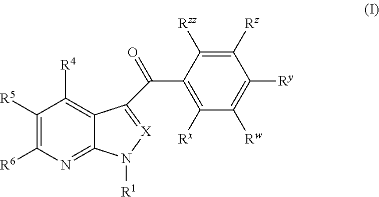
wherein
X is —CR² or N;
R¹ is H or alkyl;
R² is H or alkyl;
R⁴ is H, or alkyl;
R⁶ is H, or alkyl;
R^{w} is —NR¹⁰SO₂R¹²;
R¹⁰ is H, alkyl, or phenylalkyl;
R¹² is alkyl or haloalkyl;
R^{x}, R^{y}, R^{z} and R^{zz} are selected from:
a) R^{x} and R^{y} are halogen and R^{z} and R^{zz} are H;
b) R^{x}, R^{y} and R^{zz} are independently halogen and R^{z} is H; and
c) R^{x}, R^{y} and R^{z} are independently halogen and R^{zz} is H;
R⁵ is selected from
(a) phenyl which is substituted with 1, 2 or 3 groups independently selected from
halogen,
alkyl,
alkoxy,
alkoxy wherein the alkyl group is substituted with 1, 2 or 3 hydroxy groups, hydroxy,
—SO₂NR¹⁰R¹⁰,
—CO₂R¹⁰,
—CN,
—SF₅,
—(NR¹⁰═)S(═O)-alkyl (S-alkylsulfonimidoyl),
1H- or 2H-tetrazolyl,
—POdi(alkyl),
R¹⁰R¹⁰N—CO—,
hydroxyalkyl-ONH—CO—,
—COO—CH₂CH₂—CH(NH₂)—COOR¹⁰,
—OCH₂O— (methylenedioxy attached in neighboring positions to the phenyl ring),
—OCH₂CH₂O— (ethylenedioxy attached in neighboring positions to the phenyl ring),
a non-aromatic heterocyclic 5- or 6-membered monocyclic group having 1, 2 or 3 heteroatoms independently selected from O and N;
(b) naphthyl;
(c) a heteroaromatic 5- or 6-membered monocyclic group having 1, or 2 heteroatoms independently selected from O, N and S, wherein the heteroaromatic group is optionally substituted with 1, 2 or 3 groups independently selected from
Alkyl,
haloalkyl,
cycloalkyl,
—NR¹⁰R¹⁰,
halogen,
alkoxy, which is optionally substituted with —NR¹⁰R¹⁰
—CN,
alkenyl,
alkinyl,
R¹⁰R¹⁰N—CO—,
—(NR¹⁰═)S(═O)-alkyl,
cycloalkyl-NR¹⁰—,
alkyl-NR¹⁰—, wherein the alkyl group is substituted with hydroxy or alkoxy, alkylsulfanyl,
—COOR¹⁰,
1H- or 1H tetrazolyl,
and
a non-aromatic heterocyclic 4-, 5- or 6-membered monocyclic group having 1 or 2 heteroatoms independently selected from 0 and N, which heterocyclic group is optionally substituted with alkyl, hydroxyalkyl or hydroxy,
(d) phenyl which is fused with a heteroaromatic 5- or 6-membered monocyclic group having 1, 2 or 3 heteroatoms independently selected from O, N and S;
or a pharmaceutically acceptable salt, solvate or optical isomer thereof; and
component b) comprises at least one inhibitor selected independently from
b1) a MEK inhibitor,
b2) an ERK inhibitor, and
b3) a KRAS inhibitor, and
b4) a SHP2 inhibitor;
or a pharmaceutically acceptable salt, solvate or optical isomer thereof.

2. A composition of claim 1, wherein component a) comprises a compound of formula (I), wherein X is —CR², or a pharmaceutically acceptable salt, solvate or optical isomer thereof.

3. A composition of claim 2, wherein component a) comprises a compound of formula (I), wherein R⁵ is selected from
(a) phenyl which is substituted with 1, 2 or 3 groups independently selected from
halogen,
alkyl,
haloalkyl,
alkoxy,
alkoxy wherein the alkyl group is substituted with 1, 2 or 3 hydroxy groups,
hydroxy,
—SO₂NR¹⁰R¹⁰,
—CO₂R¹⁰,
—CN,
—SF₅,
—(NR¹⁰═)S(═O)-alkyl (S-alkylsulfonimidoyl),
1H- or 2H-tetrazolyl,
a non-aromatic heterocyclic 5- or 6-membered monocyclic group having 1, 2 or 3 heteroatoms independently selected from 0 and N;
(b) naphthyl;
(c) a heteroaromatic 5- or 6-membered monocyclic group having 1, or 2 heteroatoms independently selected from O, N and S, wherein the heteroaromatic group is optionally substituted with 1, 2 or 3 groups independently selected from
alkyl,
haloalkyl,
cycloalkyl,
—NR¹⁰R¹⁰,
halogen,
alkoxy, which is optionally substituted with —NR¹⁰R¹⁰
—CN,
alkenyl,
alkinyl,
R¹⁰R¹⁰N—CO—,
—(NR¹⁰═)S(═O)-alkyl,
cycloalkyl-NR¹⁰—,
alkyl-NR¹⁰—, wherein the alkyl group is substituted with hydroxy or alkoxy,
alkylsulfanyl,
1H- or 1H tetrazolyl,
and
a non-aromatic heterocyclic 4-, 5- or 6-membered monocyclic group having 1 or 2 heteroatoms independently selected from O and N, which heterocyclic group is optionally substituted with alkyl, hydroxyalkyl or hydroxy,
(d) phenyl which is fused with a heteroaromatic 5- or 6-membered monocyclic group having 1, 2 or 3 heteroatoms independently selected from O, N and S;
or a pharmaceutically acceptable salt, solvate or optical isomer thereof.

4. A composition of claim 3, wherein component a) comprises a compound of formula (I), wherein
R¹ is H;
R² is H;
R⁴ is H;
R⁶ is H;
R^{w} is —NR¹⁰SO₂R¹²;
R¹⁰ is H, alkyl, or phenylalkyl;
R¹² is alkyl, haloalkyl or phenylalkyl;
R^{x}, R^{y}, R^{z} and R^{zz} are selected from:
a) R^{x} and R^{y} are F and R^{z} and R^{zz} are H;
b) R^{x}, R^{y} and R^{zz} are F and R^{z} is H;
c) R^{x}, R^{z} and R^{zz} are F and R^{z} is H; and
d) R^{x}, R^{y} and R^{z} are F and R^{zz} is H;
R⁵ is selected from
(a) phenyl which is substituted with 1, 2 or 3 groups independently selected from
halogen,
alkyl,
alkoxy,
alkoxy wherein the alkyl group is substituted with 1, 2 or 3 hydroxy groups,
hydroxy,
—SO₂NR¹⁰R¹⁰,
—CO₂R¹⁰,
—CN,
—SF₅,
—(NR¹⁰═)S(═O)-alkyl (S-alkylsulfonimidoyl), and
1H- or 2H-tetrazolyl,
(b) a heteroaromatic 5- or 6-membered monocyclic group having 1, or 2 heteroatoms independently selected from O, N and S, wherein the heteroaromatic group is optionally substituted with 1, 2 or 3 groups independently selected from
alkyl,
haloalkyl,
cycloalkyl,
—NR¹⁰R¹⁰,
halogen,
alkoxy, which is optionally substituted with —NR¹⁰R¹⁰
—CN,
alkenyl,
R¹⁰R¹⁰N—CO—,
—(NR¹⁰═)S(═O)-alkyl,
cycloalkyl-NR¹⁰—,
alkyl-NR¹⁰—, wherein the alkyl group is substituted with hydroxy or alkoxy, alkylsulfanyl, and
a non-aromatic heterocyclic 4-, 5- or 6-membered monocyclic group having 1 or 2 heteroatoms independently selected from O and N, which heterocyclic group is optionally substituted with alkyl, hydroxyalkyl or hydroxy;
or a pharmaceutically acceptable salt, solvate or optical isomer thereof.

5. A composition of claim 4, wherein component a) comprises a compound of formula (I), wherein R⁵ is a heteroaromatic 5- or 6-membered monocyclic group having 1 or 2 heteroatoms independently selected from O, N and S, wherein the heteroaromatic group is optionally substituted with 1, 2 or 3 groups as defined in claim 4;
or a pharmaceutically acceptable salt, solvate or optical isomer thereof.

6. A composition of claim 5, wherein component a) comprises a compound of formula (I), wherein the heteroaromatic 5- or 6-membered monocyclic group is selected from pyridyl, pyrimidinyl, pyridazinyl and pyrazinyl and is optionally substituted with 1, 2 or 3 groups as defined in claim 4;
or a pharmaceutically acceptable salt, solvate or optical isomer thereof.

7. A composition of claim 6, wherein component a) comprises a compound of formula (I), wherein the heteroaromatic 5- or 6-membered monocyclic group is pyrimidinyl which is optionally substituted with 1 or 2 groups as defined in claim 4, and preferably with cycloalkyl;
or a pharmaceutically acceptable salt, solvate or optical isomer thereof.

8. A composition of claim 1, wherein component a) comprises a compound of formula (I), wherein X is N;
or a pharmaceutically acceptable salt, solvate or optical isomer thereof.

9. A composition of claim 8, wherein component a) comprises a compound of formula (I), wherein
R¹ is H;
R² is H;
R⁴ is H;
R⁶ is H;
R^{w} is —NR¹⁰SO₂R¹²;
R¹⁰ is H, alkyl, or phenylalkyl;
R¹² is alkyl, haloalkyl or phenylalkyl;
R^{x}, R^{y}, R^{z} and R^{zz} are selected from:
a) R^{x} and R^{y} are F and R^{z} and R^{zz} are H;
b) R^{x}, R^{y} and R^{zz} are F and R^{z} is H;
c) R^{x}, R^{z} and R^{zz} are F and R^{y} is H; and
d) R^{x}, R^{y} and R^{z} are F and R^{zz} is H;
R⁵ is selected from
(a) phenyl which is substituted with 1, 2 or 3 groups independently selected from
halogen,
alkyl,
alkoxy,
hydroxy,
—SO₂NR¹⁰R¹⁰,
—CO₂R¹⁰,
—(NR¹⁰═)S(═O)-alkyl,
1H- or 2H-tetrazolyl,
—CO—NR¹⁰R¹⁰, and
—COO—CH₂CH₂—CH(NH₂)—COOR¹⁰,
(b) a heteroaromatic 5- or 6-membered monocyclic group having 1, or 2 heteroatoms independently selected from O, N and S, wherein the heteroaromatic group is optionally substituted with 1, 2 or 3 groups independently selected from
alkyl,
haloalkyl,
cycloalkyl,
halogen,
alkoxy,
—CN,
hydroxy,
alkylsulfanyl,
—COOR¹⁰, and
1H- or 1H tetrazolyl;
or a pharmaceutically acceptable salt, solvate or optical isomer thereof.

10. A composition of claim 9, wherein component a) comprises a compound of formula (I), wherein R⁵ is a heteroaromatic 5- or 6-membered monocyclic group having 1, or 2 heteroatoms independently selected from O, N and S, wherein the heteroaromatic group is optionally substituted with 1, 2 or 3 groups as defined in claim 9;
or a pharmaceutically acceptable salt, solvate or optical isomer thereof.

11. A composition of claim 10, wherein component a) comprises a compound of formula (I), wherein the heteroaromatic 5- or 6-membered monocyclic group is selected from pyridyl and pyrimidinyl and is optionally substituted with 1, 2 or 3 groups as defined in claim 9;
or a pharmaceutically acceptable salt, solvate or optical isomer thereof.

12. A composition of claim 11, wherein component a) comprises a compound of formula (I), wherein the heteroaromatic 5- or 6-membered monocyclic group is unsubstituted pyridyl or pyrimidinyl which is optionally substituted with 1 or 2 groups as defined in claim 9, and preferably with cycloalkyl;
or a pharmaceutically acceptable salt, solvate or optical isomer thereof.

13. A composition of any one of claims 1 to 12, wherein component b) comprises a compound selected from 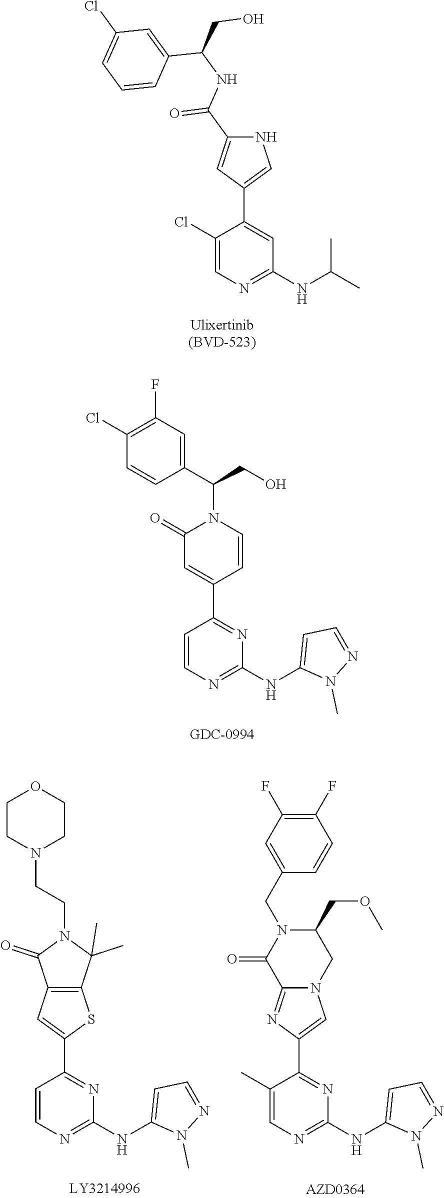 [SMILES: CC(C)Nc1cc(-c2c[nH]c(C(=O)N[C@H](CO)c3cccc(Cl)c3)c2)c(Cl)cn1 ; Cn1nccc1Nc1nccc(-c2ccn([C@H](CO)c3ccc(Cl)c(F)c3)c(=O)c2)n1 ; Cn1nccc1Nc1nccc(-c2cc3c(s2)C(C)(C)N(CCN2CCOCC2)C3=O)n1 ; COC[C@H]1Cn2cc(-c3nc(Nc4ccnn4C)ncc3C)nc2C(=O)N1Cc1ccc(F)c(F)c1] 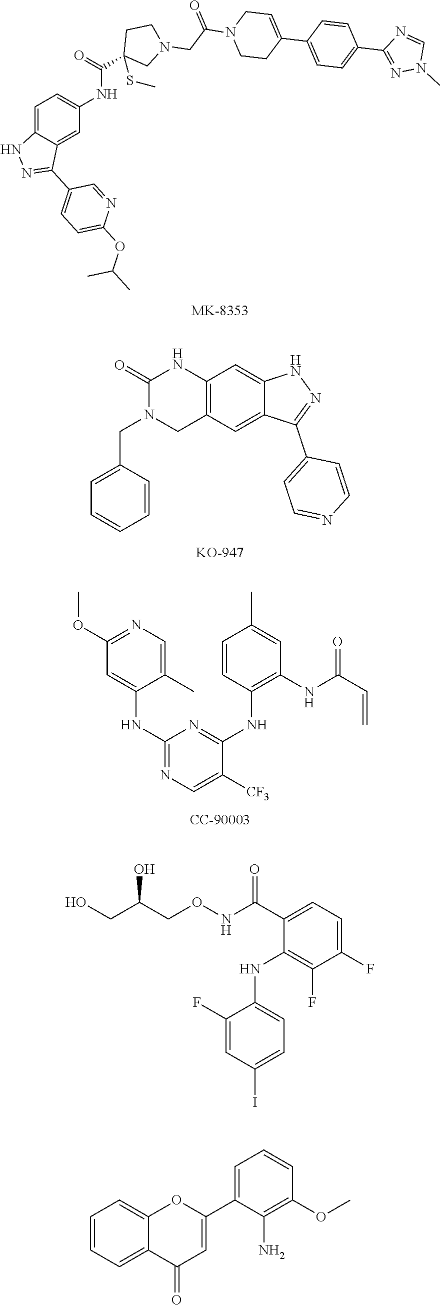 [SMILES: CS[C@@]1(C(=O)Nc2ccc3[nH]nc(-c4ccc(OC(C)C)nc4)c3c2)CCN(CC(=O)N2CC=C(c3ccc(-c4ncn(C)n4)cc3)CC2)C1 ; O=C1Nc2cc3[nH]nc(-c4ccncc4)c3cc2CN1Cc1ccccc1 ; C=CC(=O)Nc1cc(C)ccc1Nc1nc(Nc2cc(OC)ncc2C)ncc1C(F)(F)F ; O=C(NOC[C@H](O)CO)c1ccc(F)c(F)c1Nc1ccc(I)cc1F ; COc1cccc(-c2cc(=O)c3ccccc3o2)c1N] 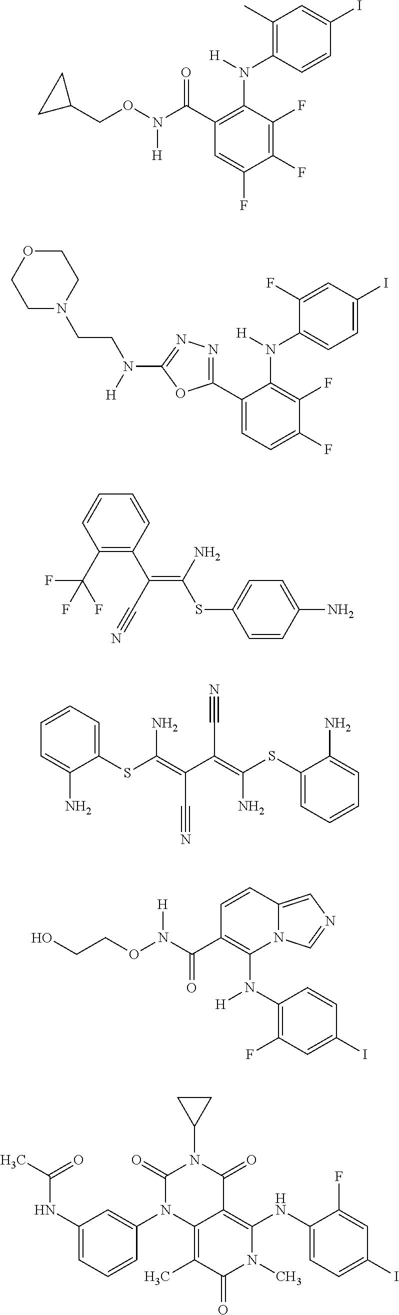 [SMILES: Cc1cc(I)ccc1Nc1c(C(=O)NOCC2CC2)cc(F)c(F)c1F ; Fc1cc(I)ccc1Nc1c(-c2nnc(NCCN3CCOCC3)o2)ccc(F)c1F ; N#C/C(=C(/N)Sc1ccc(N)cc1)c1ccccc1C(F)(F)F ; N#CC(=C(/N)Sc1ccccc1N)/C(C#N)=C(\N)Sc1ccccc1N ; O=C(NOCCO)c1ccc2cncn2c1Nc1ccc(I)cc1F ; CC(=O)Nc1cccc(-n2c(=O)n(C3CC3)c(=O)c3c(Nc4ccc(I)cc4F)n(C)c(=O)c(C)c32)c1] 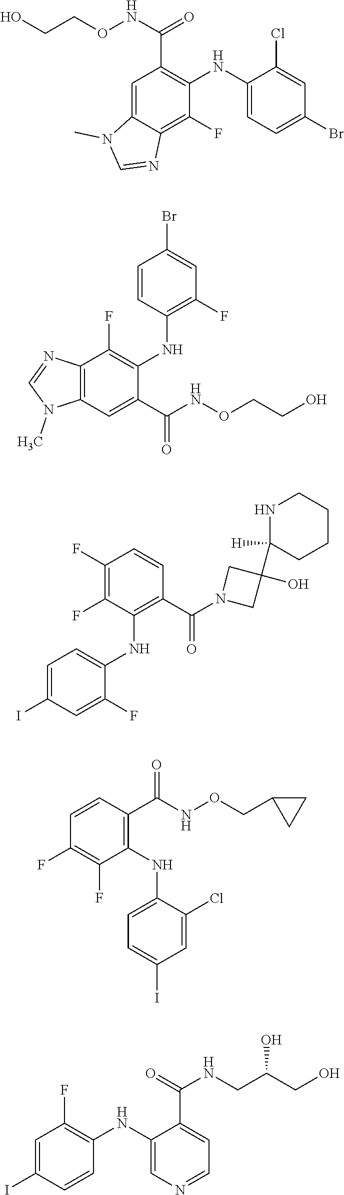 [SMILES: Cn1cnc2c(F)c(Nc3ccc(Br)cc3Cl)c(C(=O)NOCCO)cc21 ; Cn1cnc2c(F)c(Nc3ccc(Br)cc3F)c(C(=O)NOCCO)cc21 ; O=C(c1ccc(F)c(F)c1Nc1ccc(I)cc1F)N1CC(O)([C@@H]2CCCCN2)C1 ; O=C(NOCC1CC1)c1ccc(F)c(F)c1Nc1ccc(I)cc1Cl ; O=C(NC[C@H](O)CO)c1ccncc1Nc1ccc(I)cc1F] 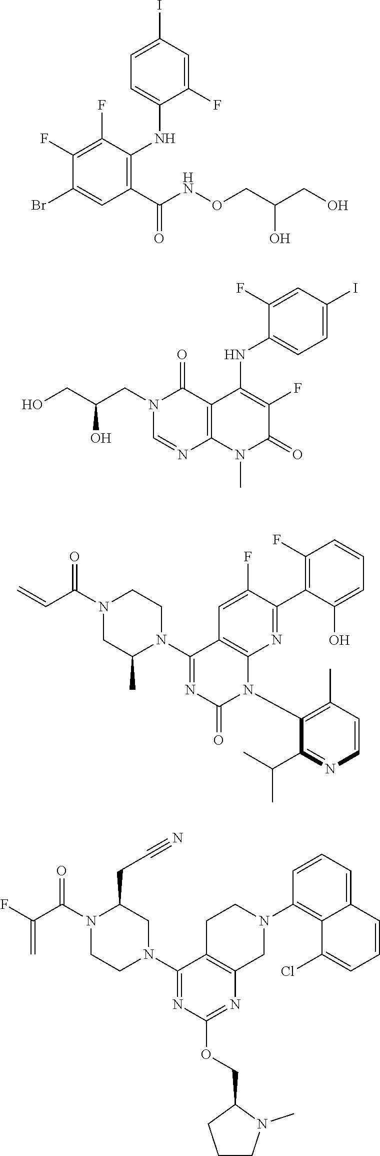
or a pharmaceutically acceptable salt, solvate or optical isomer thereof.

14. A composition of claim 13, wherein
component a) comprises a compound selected from
N-(3-(5-(2-cyclopropyl-pyrimidin-5-yl)-1H-pyrrolo[2,3-b]pyridine-3-carbonyl)-2,6-difluorophenyl)-propane-1-sulfonamide, N-(2,6-difluoro-3-(5-(pyridin-4-yl)-1H-pyrazolo[3,4-b]pyridine-3-carbonyl)phenyl)propane-1-sulfonamide, and
N-(3-(5-(2-cyclopropylpyrimidin-5-yl)-1H-pyrazolo[3,4-b]pyridine-3-carbonyl)-2,6-difluorophenyl)propane-1-sulfonamide; or a pharmaceutically acceptable salt, solvate or optical isomer thereof; and
component b) comprises a compound selected from trametinib, SCH772984, sotorasib and RMC4550; or a pharmaceutically acceptable salt, solvate or optical isomer thereof.

15. A compound of formula I as defined in any one of claims 1 to 12 or 14 or a pharmaceutically acceptable salt, solvate or optical isomer thereof for use in treating cancer, preferably colon or lung cancer and in particular KRAS mutant colon and KRAS mutant lung cancer.
